(19) United States
(12) Patent Application Publication
(10) Pub. No.: US 2024/0268683 A1
(43) Pub. Date: Aug. 15, 2024

(54) SYSTEM AND METHOD FOR MONITORING INFANT FEEDING

(71) Applicant: THE JOHNS HOPKINS UNIVERSITY, Baltimore, MD (US)

(72) Inventors: Danielle Gottlieb SEN, Baltimore, MD (US); Ignacio ALBERT, Madrid (ES); Summer DUFFY, Baltimore, MD (US); Kate CALLIGY, Rosedale, MD (US); Matthew BANET, San Diego, CA (US)

(21) Appl. No.: 18/438,189

(22) Filed: Feb. 9, 2024

Related U.S. Application Data
(60) Provisional application No. 63/484,754, filed on Feb. 14, 2023.

(51) Int. Cl. A61B 5/0205; A61B 5/00; A61B 5/024; A61B 5/0245; A61B 5/08; A61B 5/091; A61B 5/1455; A61B 5/282; A61B 5/304; A61B 5/308; A61B 5/346

(52) U.S. Cl. CPC A61B 5/0205; A61B 5/282; A61B 5/304; A61B 5/308; A61B 5/346; A61B 5/6808; A61B 5/02405; A61B 5/0245; A61B 5/0816; A61B 5/091; A61B 5/14552; A61B 2503/04; A61B 2560/0214; A61B 2562/0219; A61B 2562/164

(57) ABSTRACT

A system for monitoring a patient includes a first drive electrode configured to be in contact with the patient. The first drive electrode is configured to receive a first electrical current and inject it into the patient. The system also includes a first sense electrode configured to be in contact with the patient. The first sense electrode is configured to sense a first bio-electric signal from the patient. The first bio-electric signal is modified by the first electrical current. The system also includes an impedance circuit connected to the first drive electrode and the first sense electrode. The impedance circuit is configured to measure a bio-impedance or bio-reactance waveform in response to the first bio-electric signal. The system also includes a physiological sensor connected to the first drive electrode and the first sense electrode. The physiological sensor is configured to measure an electrocardiogram waveform based upon the first bio-electric signal.

### CROSS-REFERENCE TO RELATED APPLICATIONS

This application claims priority to U.S. Provisional Patent Application No. 63/484,754, filed on Feb. 14, 2023, which is incorporated by reference.

### FIELD OF THE DISCLOSURE

The present disclosure relates generally to systems and methods for monitoring an infant. More particularly, the present disclosure relates to systems and methods for monitoring an infant's feeding, urine, feces, sleep, health, and/or wellness.

### BACKGROUND OF THE DISCLOSURE

3.5 million babies are born in the U.S. every year who eat, excrete, and grow. Proper nutrition is important for infant development, especially for premature and low-weight infants. Nutrition also plays a role in the overall health of the infant. 8.22 million Americans used liquid/powdered infant formula, most of which is fed through bottles. Breastfeeding also plays a role in infant feeding, with 82% of mothers reporting breastfeeding their children. The breastfeeding percentages decrease to 61% at 6 months, and 34% at 1 year. Mothers generally wear bras, in particular nursing bras, during breastfeeding. Mothers and caretakes have difficulties evaluating whether the infant has been sufficiently fed, especially during breastfeeding, when there is no way to track the amount of milk other than weight the infant before and after breastfeeding, which is time-consuming and tedious.

Infant's sleep quality and cycles are also hard to monitor for the caretaker, whose own sleep schedule can depend on the sleep of the infant. In addition, vital sign monitoring of infants is important to assess their state of health and wellbeing. Conventional infant monitoring systems typically include a crib-side camera and microphone for capturing images and sounds generated by the infant, and a 1-way wireless system for transmitting these images to a remote display that can be viewed by a family member (e.g., a parent). With such a system, the parent can be removed from the crib and still determine whether the infant is sleeping, crying, or moving about. Typically, such systems include viewing devices that are custom-made, hand-held, and feature a simple display for rendering images of the infant and a speaker system for projecting their sounds.

What is needed is an improved system and method for monitoring an infant (e.g., monitoring an infant's feeding, urine, feces, sleep, health, and/or wellness).

### SUMMARY

A system for monitoring a patient is disclosed. The system includes a first drive electrode configured to be in contact with the patient. The first drive electrode is configured to receive a first electrical current and inject it into the patient. The system also includes a first sense electrode configured to be in contact with the patient. The first sense electrode is configured to sense a first bio-electric signal from the patient. The first bio-electric signal is modified by the first electrical current. The system also includes an impedance circuit connected to the first drive electrode and the first sense electrode. The impedance circuit is configured to measure a bio-impedance or bio-reactance waveform in response to the first bio-electric signal. The system also includes a physiological sensor connected to the first drive electrode and the first sense electrode. The physiological sensor is configured to measure an electrocardiogram (ECG) waveform based upon the first bio-electric signal. The system also includes a computing system configured to determine a respiration rate and/or a respiratory tidal volume of the patient based upon the bio-impedance or bio-reactance waveform. The computing system is also configured to determine a heart rate and/or a heart rate variability of the patient based upon the ECG waveform.

A system for monitoring a patient wearing a diaper is also disclosed. The system includes a flexible substrate configured to be worn between the diaper and the patient. The flexible substrate includes a first drive electrode configured to be in contact with the patient. The first drive electrode is configured to receive a first electrical current and inject it into the patient. The system also includes a second drive electrode configured to be in contact with the patient. The second drive electrode is configured to receive a second electrical current and inject it into the patient. The system also includes a first sense electrode configured to be in contact with the patient. The first sense electrode is configured to sense a first bio-electric signal from the patient. The first bio-electric signal is modified by the first electrical current. The system also includes a second sense electrode configured to be in contact with the patient. The second sense electrode is configured to sense the first bio-electric signal and a second bio-electric signal from the patient. The second bio-electric signal is modified by the second electrical current. The system also includes an impedance circuit connected to the first and second drive electrodes and the first and second sense electrodes. The impedance circuit is configured to measure a bio-impedance or bio-reactance waveform in response to the first and second bio-electric signals. The system also includes a physiological sensor connected to the first drive electrode and the first sense electrode. The physiological sensor is configured to measure an electrocardiogram (ECG) waveform based upon the first bio-electric signal. The system also includes a computing system configured to determine a respiration rate and/or a respiratory tidal volume of the patient based upon the bio-impedance or bio-reactance waveform. The computing system is also configured to determine a heart rate and/or a heart rate variability of the patient based upon the ECG waveform.

In another embodiment, the system includes an impedance circuit. The system also includes a multiplex circuit in electrical contact with the impedance circuit and configured to couple the impedance circuit to a physiological sensor and, at a later time, to a waste-monitoring sensor. The physiological sensor is in electrical contact with the multiplex circuit and in physical contact with the patient and includes a first drive electrode configured to receive a first electrical current from the impedance circuit and inject it into the patient, and a first sense electrode configured to sense a bio-electric signal from the patient, with the bio-electric signal being modified by the first electrical current. The system also includes a processing system in electrical contact with the impedance circuit and the multiplex circuit. The processing system is configured to: i) control the multiplex circuit to couple the impedance circuit to either the physiological sensor or the waste-monitoring sensor; ii) when the impedance circuit is coupled to the physiological sensor, process the bio-electric signal or a signal derived therefrom to measure a physiological property from the patient; and iii) when the impedance circuit is coupled to the waste-monitoring sensor, process the waste signal or a signal derived therefrom to determine a waste property from the patient.

A system for monitoring a patient wearing a diaper is also disclosed. The system includes a flexible substrate configured to be worn between the diaper and the patient. The flexible substrate includes an impedance circuit. The flexible substrate also includes a multiplex circuit in electrical contact with the impedance circuit and configured to couple the impedance circuit to a physiological sensor and, at a later time, to a waste-monitoring sensor. The physiological sensor is in electrical contact with the multiplex circuit and in physical contact with the patient and includes a first drive electrode configured to receive a first electrical current from the impedance circuit and inject it into the patient, and a first sense electrode configured to sense a bio-electric signal from the patient, with the bio-electric signal being modified by the first electrical current. The waste-monitoring sensor is in electrical contact with the multiplex circuit and in physical contact with a region proximal to an anus of the patient and a genitalia of the patient. The waste-monitoring sensor includes a second drive electrode configured to receive a second electrical current generated by the impedance circuit and inject it into the region, and a second sense electrode configured to sense a waste signal from the region, with the waste signal being modified by the second electrical current. The system also includes a processing system in electrical contact with the impedance circuit and the multiplex circuit. The processing system is configured to: i) control the multiplex circuit to couple the impedance circuit to either the physiological sensor or the waste-monitoring sensor; ii) when the impedance circuit is coupled to the physiological sensor, process the bio-electric signal or a signal derived therefrom to measure a physiological property from the patient; and iii) when the impedance circuit is coupled to the waste-monitoring sensor, process the waste signal or a signal derived therefrom to determine a waste property from the patient.

A system for monitoring a patient wearing a diaper is also disclosed. The system includes a flexible substrate configured to be worn between the diaper and the patient that includes an impedance circuit. The flexible substrate also includes a multiplex circuit. The flexible substrate also includes a physiological sensor in electrical contact with the multiplex circuit and in physical contact with the patient and configured to sense a bio-electric signal from the patient when coupled to the multiplex circuit. The flexible substrate also includes a waste-monitoring sensor in electrical contact with the multiplex circuit and in physical contact with a region proximal to an anus of the patient and a genitalia of the patient and configured to sense a waste signal from the region when coupled to the multiplex circuit. The system also includes a processing system configured to control the impedance circuit and the multiplex circuit to sequentially detect the bio-electric signal and the waste signal.

A nursing bottle is also disclosed. The nursing bottle includes a bottle defining an internal volume configured to receive milk or infant formula. The nursing bottle also includes one or more sensors coupled to the bottle that are configured to measure one or more parameters. The nursing bottle also includes a computing system configured to receive the one or more parameters and to determine an amount of the milk or infant formula remaining in the bottle, an amount of the milk or infant formula consumed from the bottle by an infant, or both based at least partially upon the one or more parameters.

A nursing bra is also disclosed. The nursing bra includes a first cup configured to receive a first breast of a nursing mother. The nursing bra also includes a second cup configured to receive a second breast of the nursing mother. The nursing bra also includes a center gore coupled to and positioned between the first and second cups. The nursing bra also includes a first strap coupled to the first cup and configured to extend over a first shoulder of the nursing mother. The nursing bra also includes a second strap coupled to the second cup and configured to extend over a second shoulder of the nursing mother. The nursing bra also includes one or more sensors coupled to the nursing bra that are configured to measure one or more parameters. The nursing bra also includes a computing system configured to receive the one or more parameters and to determine an amount of the milk consumed by an infant from the first breast, the second breast, or both based at least partially upon the one or more parameters.

A pacifier is also disclosed. The pacifier includes a handle. The pacifier also includes a mouth shield coupled to the handle. The pacifier also includes a teat coupled to the mouth shield. The teat is configured to be positioned in a mouth of an infant. The pacifier also includes one or more sensors positioned at least partially within the teat. The one or more sensors are configured to measure a temperature of the infant.

### BRIEF DESCRIPTION OF THE FIGURES

FIG. 1 is a flowchart of a method for monitoring an infant using one or more sensor devices, according to an embodiment.

FIG. 2 is a schematic view of the sensor devices that may be used to perform at least a portion of the method in FIG. 1, according to an embodiment.

FIG. 3 is a flowchart of a method for monitoring an infant's sleep and/or health, according to an embodiment.

FIG. 4 is a schematic view of a system that may be used to perform at least a portion of the method in FIG. 3, according to an embodiment.

FIG. 5 is a flowchart of a method for monitoring an infant's sleep, according to an embodiment.

FIG. 6 is a schematic diagram of multiple configurations of infant and maternal monitors connected to each other and to a notification application, according to an embodiment.

FIG. 7 is a table containing a variety of devices that can evaluate an infant's feeding, body assessment, and/or output, according to an embodiment.

FIG. 8 is a flowchart of a method for calibrating a smart bra or smart bottle using a weight scale during infant feeding, according to an embodiment.

FIG. 9 is a schematic diagram of feeding-tracking and quantification devices that may be used to perform at least a portion of the method in FIG. 8, according to an embodiment.

FIG. 10 is a flowchart of a method for monitoring an infant's nutrition, according to an embodiment.

FIG. 11 is a flowchart of a method for monitoring an infant's excretion and/or hydration using one or more sensor devices, according to an embodiment.

FIG. 12 is a schematic view of the sensor devices that may be used to perform at least a portion of the method in FIG. 11, according to an embodiment.

FIG. 13 is a flowchart of a method for monitoring an infant's nutrition, growth, and/or development using one or more sensor devices, according to an embodiment.

FIG. 14 is a schematic view of a wireless connected monitoring system for monitoring an infant's nutrition, according to an embodiment.

FIG. 15A is a schematic side view of a feeding bottle with photodetectors to measure the amount of milk or formula in the bottle and/or consumed by the infant, according to an embodiment.

FIG. 15B is a schematic side view of the feeding bottle with pressure sensors to measure the amount of milk or formula in the bottle and an accelerometer to measure orientation of the bottle, according to an embodiment.

FIG. 15C is a schematic side view of the feeding bottle with bioimpedance electrodes to measure the amount of milk or formula in the bottle, according to an embodiment.

FIG. 15D is a schematic side view of the feeding bottle with a buoyant ball attached to a spring to measure the amount of milk or formula in the bottle, according to an embodiment.

FIG. 16A is a schematic side view of a smart feeding bottle that measures the amount of milk or formula consumed by the infant where the processing and communications module is in the bottle, according to an embodiment.

FIG. 16B is a schematic side view of the smart feeding bottle that measures the amount of milk or formula consumed by the infant where the processing and communications module is in the cap of the bottle, according to an embodiment.

FIG. 17A is a schematic view of a biosensor diaper patch, according to an embodiment.

FIG. 17B is a perspective view of the biosensor patch placed on a diaper, according to an embodiment.

FIG. 18A is a front view of a smart bra that measures the amount of milk delivered during breastfeeding using bioimpedance, according to an embodiment.

FIG. 18B is a front view of a smart bra that measures the amount of milk delivered during breastfeeding using bioimpedance, according to an embodiment.

FIG. 18C is a front view of a smart bra that measures the amount of milk delivered during breastfeeding using pressure sensors in the cups or underwire, according to an embodiment.

FIG. 18D is a front view of a smart bra that measures the amount of milk delivered during breastfeeding using pressure sensors in the straps, according to an embodiment.

FIG. 18E is a front view of a smart nursing bra that measures the amount of milk delivered during breastfeeding using bioimpedance sensors, according to an embodiment.

FIG. 18F is a front view of a smart nursing bra that measures the amount of milk delivered during breastfeeding using bioimpedance sensors, according to an embodiment.

FIG. 19 is a schematic view of a smart pad insert that measures the amount of milk delivered during breastfeeding, according to an embodiment.

FIG. 20A is a front view (facing towards the body) of a wearable sensor insert worn within a diaper or onesie, according to an embodiment.

FIG. 20B is a back view (facing away from the body) of the wearable sensor insert worn inside the diaper or onesie, according to an embodiment.

FIG. 21A is a perspective view of a disposable electronic smart diaper, according to an embodiment.

FIG. 21B is a back view of a reusable case clipped to the disposable electronic diaper, according to an embodiment.

FIG. 21C is a front view of the reusable case clipped to the disposable electronic diaper, according to an embodiment.

FIG. 21D is a bottom view of the reusable case clipped to the disposable electronic diaper, according to an embodiment.

FIG. 22A is a side view of a smart pacifier with sensors in the surface for measuring nutrition-related information from saliva and temperature, according to an embodiment.

FIG. 22B is a side view of a smart pacifier with sensors inside the pacifier nipple for measuring nutrition-related information from saliva and temperature, according to an embodiment.

FIG. 23 is a front view of a smart monitoring onesie, according to an embodiment.

FIG. 24A is a front view of a flexible, stretchable, smart waistband worn by an infant, according to an embodiment.

FIG. 24B is a back view of the flexible, stretchable, smart waistband worn by the infant, according to an embodiment.

FIG. 25A is a side view of a weight and bioimpedance scale embodied in a changing pad that can measure weight and/or body composition of the infant, according to an embodiment.

FIG. 25B is a top cross-sectional view of the weight and bioimpedance scale, according to an embodiment.

FIG. 26A is a side view of a weight and bioimpedance scale embodied in a stroller that can measure weight and body composition of the infant, according to an embodiment.

FIG. 26B is a side view of a weight and bioimpedance scale embodied in a bassinet that can measure weight and body composition of the infant, according to an embodiment.

FIG. 26C is a side view of a weight and bioimpedance scale embodied in crib that can measure weight and body composition of the infant, according to an embodiment.

FIG. 27A is a front view of a smart bra that measures the amount of milk delivered during breastfeeding using bioimpedance, according to an embodiment.

FIG. 27B is a back view of the smart bra that measures the amount of milk delivered during breastfeeding using bioimpedance, according to an embodiment.

FIG. 27C is a left-side view of the smart bra that measures the amount of milk delivered during breastfeeding using bioimpedance, according to an embodiment.

FIG. 27D is a right-side view of the smart bra that measures the amount of milk delivered during breastfeeding using bioimpedance.

FIGS. 28A-28D are examples of displays (e.g., dashboard views) that may be generated by one or more of the devices herein (e.g., the wearable sensor insert).

FIG. 29A is a graph showing voltage versus time for the chest.

FIG. 29B is a graph showing voltage versus time for the abdomen.

FIG. 30A is a graph showing ECG versus time.

FIG. 30B is a graph showing impedance versus time.

FIG. 31A is a schematic drawing of a front side (facing towards the body) of a wearable sensor insert worn within a diaper or onesie, FIG. 31B is a schematic drawing of a back side (facing away from the body) of the wearable sensor insert worn inside a diaper or onesie, and FIG. 31C is a schematic drawing of an inside of a casing of the wearable sensor insert worn inside a diaper or onesie, according to an embodiment.

FIG. 32 illustrates a schematic view of an infant wearing a wearable sensor on the abdomen/belly, according to an embodiment.

FIG. 33 illustrates a schematic view of a wireless, connected monitoring system for a patient (e.g., infant) that integrates to a patient monitoring system, according to an embodiment.

FIG. 34 illustrates a flowchart of a method for monitoring a patient, according to an embodiment.

FIG. 35 illustrates a table listing monitoring methods in the system with associated physiological signals.

### DETAILED DESCRIPTION

### Connected Devices for Caretaker-Infant Monitoring and Notification

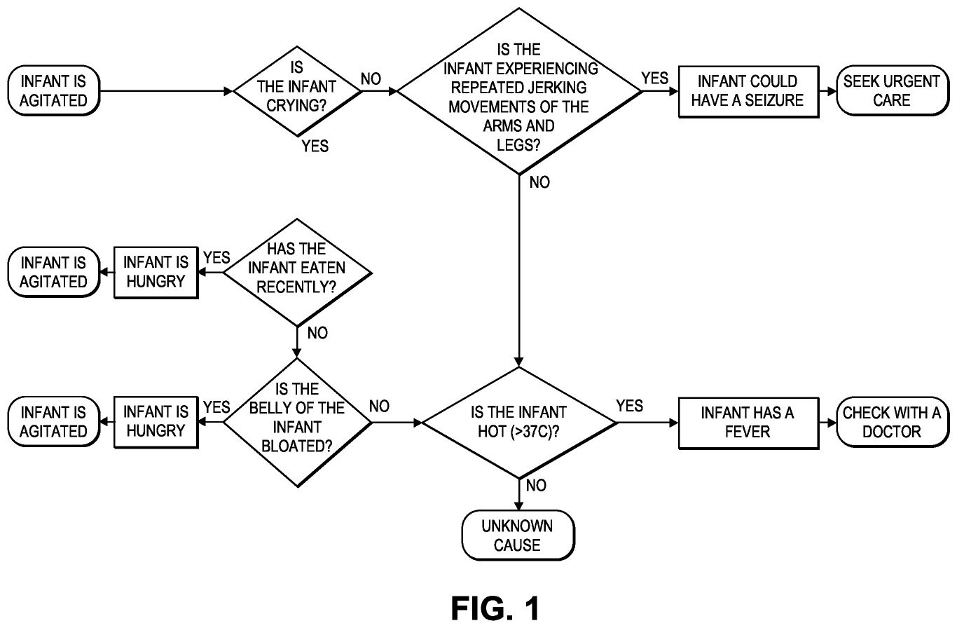

FIG. 1 is a flowchart of a method for monitoring an infant using one or more sensor devices, according to an embodiment. As shown, the method may help to determine whether the infant is hungry, is suffering from colic, has a fever, may have a seizure, or a combination thereof. The method may be performed with the help of one or more sensor devices, which are described below.

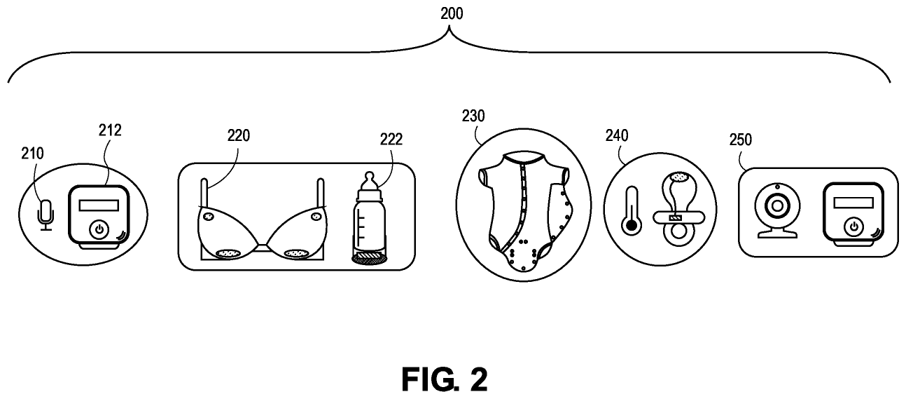

FIG. 2 is a schematic view of a system 200 including one or more sensor devices that may be used to perform at least a portion of the method in FIG. 1, according to an embodiment. The system 200 may include a microphone 210 and/or camera 212. The microphone 210 and/or camera 212 may be or include stationary (e.g., crib-side) devices, or wearable devices that may be attached to a diaper or clothing (e.g., onesie). The system 200 may also include a smart bra 220 that may measure and/or quantify the amount of breast milk consumed by the infant. The system 200 may also include a smart bottle 222 that may measure and/or quantify the amount of milk or formula consumed by the infant. The system 200 may also include a smart clothing item (e.g., a onesie) 230 with one or more sensors (e.g., bioimpedance sensors) to measure abdominal bloating. The system 200 may also include a smart pacifier 240 that includes a thermometer to measure the temperature of the infant. The system 200 may also include a movement sensor 250, which may be or include a video camera, or wearable device that includes an accelerometer and/or gyroscope.

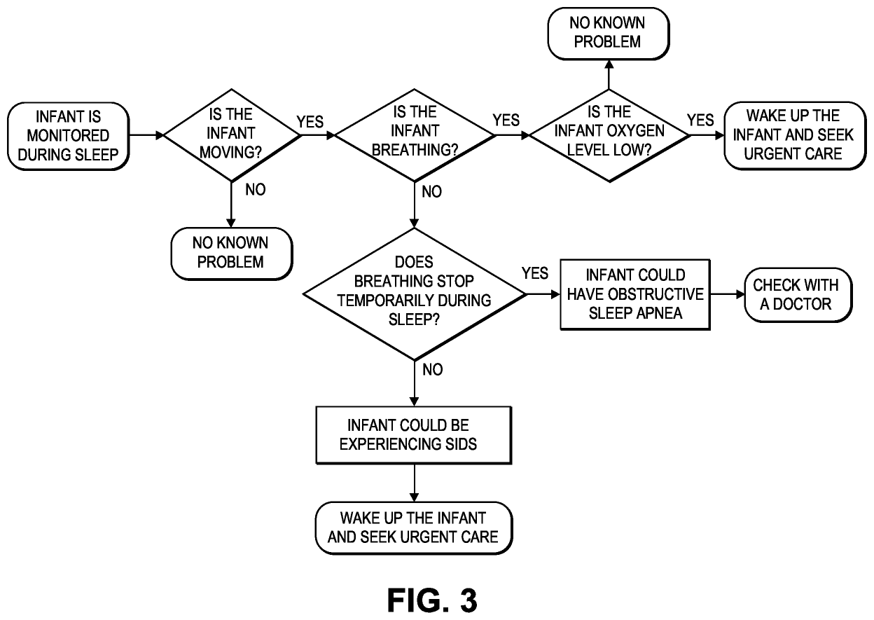

FIG. 3 is a flowchart of a method for monitoring an infant's sleep and/or health, according to an embodiment. As shown, the method may help to determine whether the infant may be experiencing sudden infant death syndrome (SIDS), a low oxygen level, obstructive sleep apnea, or a combination thereof.

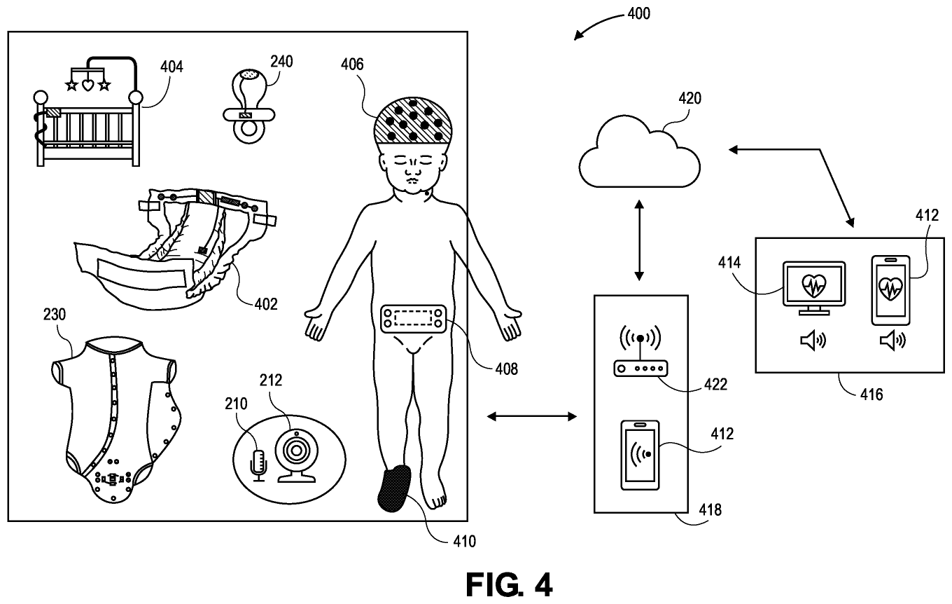

FIG. 4 is a schematic view of a system 400 for performing at least a portion of the method in FIG. 3, according to an embodiment. The system 400 may at least partially overlap with the system 200. The system 400 may include the microphone 210 and/or camera 212 for crib-side monitoring. The system 400 may also include the smart clothing item (e.g., a onesie 230). The system 400 may also include the smart pacifier 240. The system 400 may also include an infant EEG sensor 406. The system 400 may also include a smart sock 410 with sensors (e.g., electrodes) configured to measure to measure heart rate, SpO2, and/or peripheral body temperature. The system 400 may also include a smartphone 412 to access and display data measured by one or more of the devices described above. The system 400 may also include a computer 414 to access and display data. The system 400 may also include a visual and audio data display 416 that is configured to generate and/or display graphs, charts, and/or alarms based upon the measured data. The system 400 may also include a gateway 418 to interface between the aforementioned devices and a cloud data storage 420. The system 400 may be configured to transmit the data via a router 422 or via Bluetooth, BLE, etc. The system 400 may also include an infant wearable vital sign sensor 408, that sits below the diaper strap and collects ECG, Bio-Z, PPG, and more.

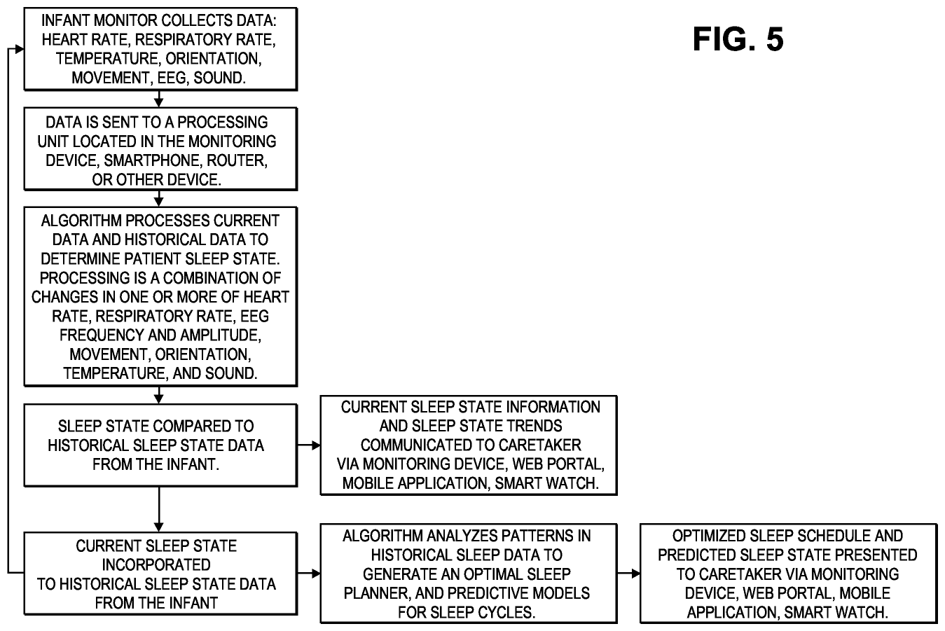

FIG. 5 is a flowchart of a method for monitoring an infant's sleep, according to an embodiment. As shown, the method may determine current sleep state information and trends. The method may also or instead determine or predict an optimized sleep schedule and/or sleep state for the infant.

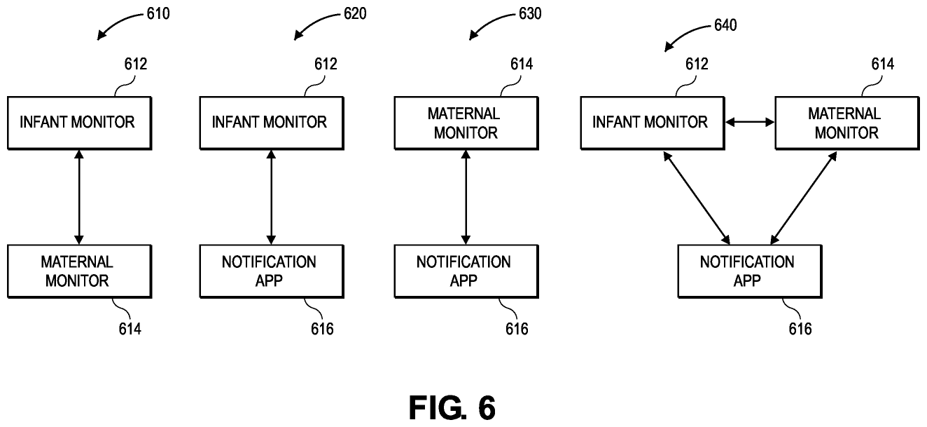

FIG. 6 is a schematic diagram of multiple configurations of infant and maternal monitors connected to each other and to a notification application, according to an embodiment. A first configuration 610 includes an infant monitoring system 612 and maternal monitoring system 614 connected to each other. A second configuration 620 includes the infant monitoring system 612 connected to a notification application 616 (e.g., in a smartphone). A third configuration 630 includes the maternal monitoring system 614 connected to the notification application 616. A fourth configuration 640 includes the infant monitoring system 612 and maternal monitoring system 614 connected to each other, and to the notification application 616 that can display information to the user related to the data obtained the monitor, and present guidelines or recommendations on how to act on this information.

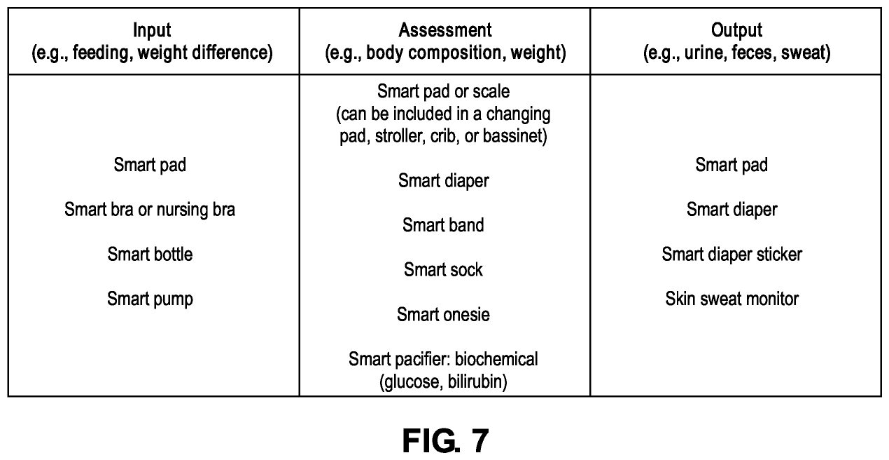

FIG. 7 is a table containing a variety of smart connect devices that can evaluate an infant's feeding, body assessment, and/or output, according to an embodiment.

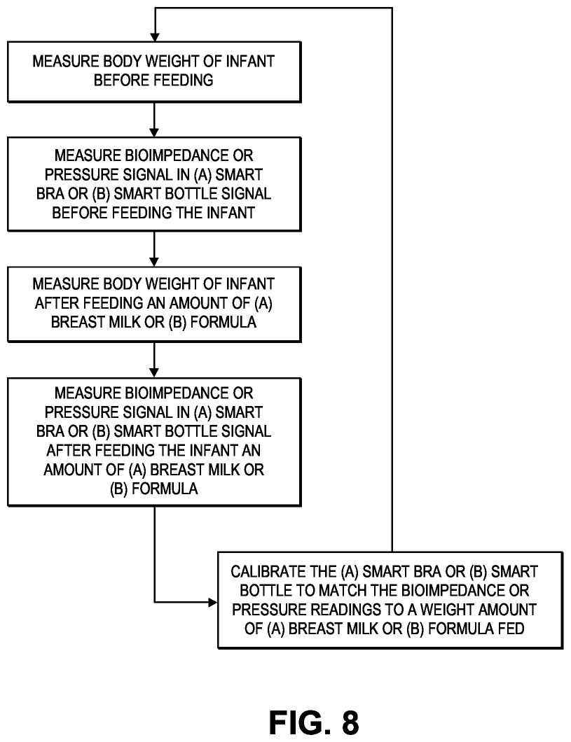

FIG. 8 is a flowchart of a method for calibrating the smart bra 220 and/or smart bottle 222 using a weight scale during infant feeding, according to an embodiment. As shown, the method may be used to calibrate the smart bra 220 and/or smart bottle 222 to match the bioimpedance and/or pressure measurements to an amount of breast milk and/or formula. The method may be iterative (e.g., repeated) to obtain multiple measurements, which may help to reduce error and determine a calibration curve.

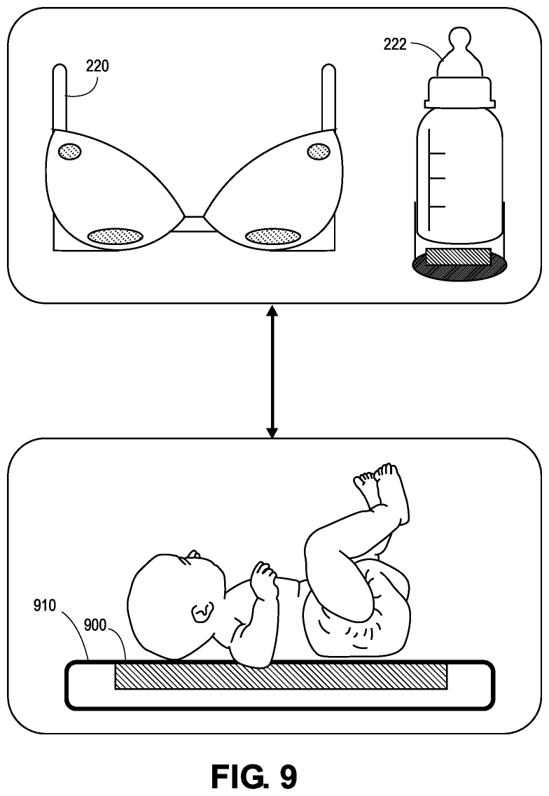

FIG. 9 is a schematic diagram of devices that may be used to perform a portion of the method shown in FIG. 8, according to an embodiment. More particularly, FIG. 9 includes feeding-tracking and quantification devices connected to a smart infant weight scale 900 or changing pad 910, according to an embodiment. The bra 220 can measure the bioimpedance of the mother's breast(s) and/or the pressure within the breast(s). The bottle 222 can measure the pressure within the bottle 222. The bra 220 and/or bottle 222 can be connected to the scale 900 and/or changing pad 910, which is/are configured to measure the infant's weight. For example, the infant's weight may be measured before and/or after feeding to determine how much the infant's weight has increased (e.g., how much milk and/or formula the infant has consumed). The smart bra 220, the bottle 222, the scale 900, and/or the pad 910 may transmit measurements back and forth to correlate and/or calibrate the bioimpedance and/or pressure measurements to the amount of breast milk and/or formula consumed.

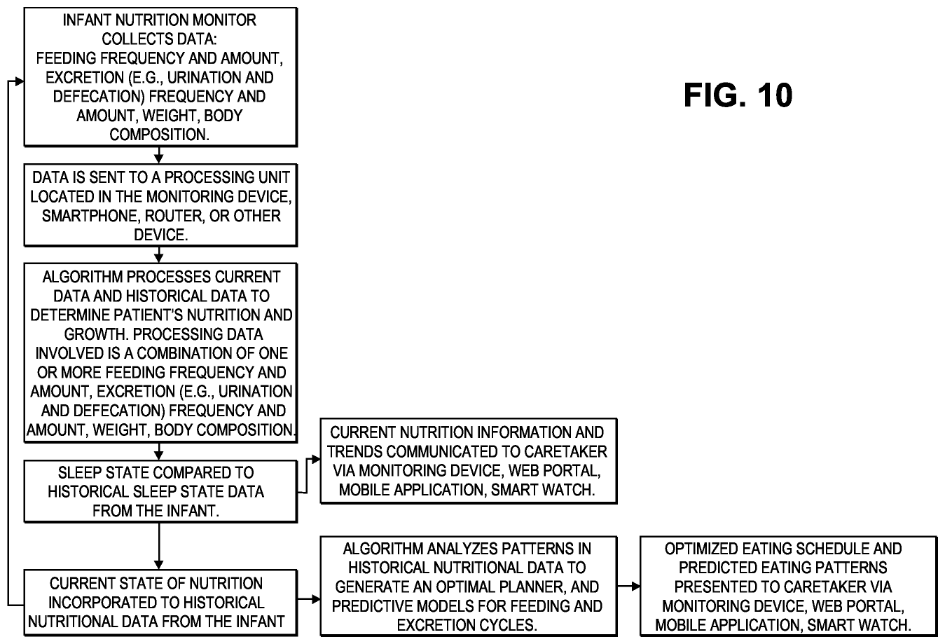

FIG. 10 is a flowchart of a method for monitoring a nutrition of an infant, according to an embodiment. As shown, the method may determine the current nutrition information and/or nutrition trends for the infant. The method may also or instead determine an optimized eating schedule and/or optimized eating patterns for the infant. The method may be iterative (e.g., repeated) to obtain multiple measurements, which may help to reduce error.

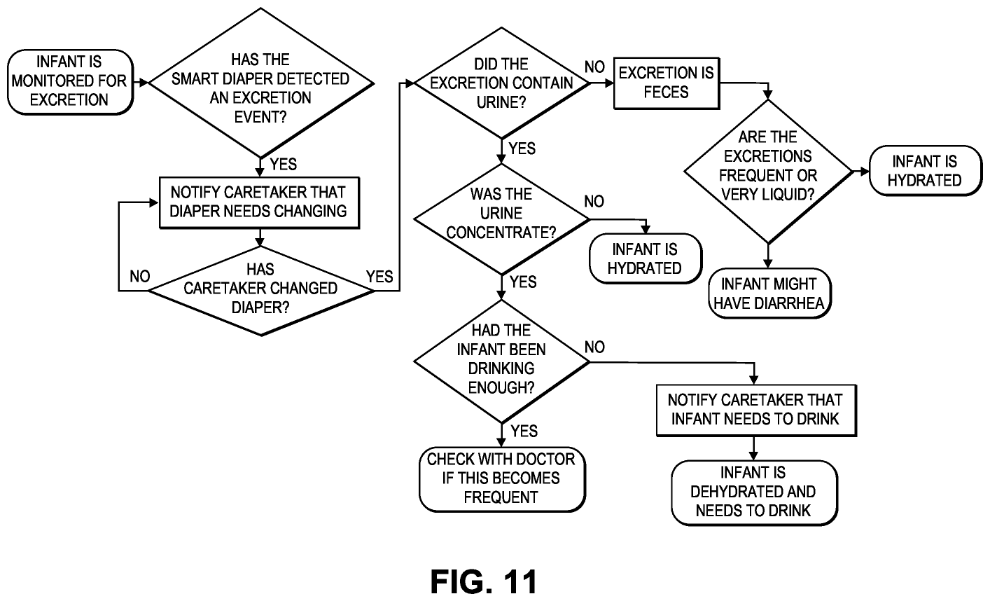

FIG. 11 is a flowchart of a method for monitoring an infant's excretion and/or hydration using one or more sensor devices, according to an embodiment. As shown, the method may be used to determine whether the infant is hydrated or dehydrated, and/or whether the infant may have diarrhea.

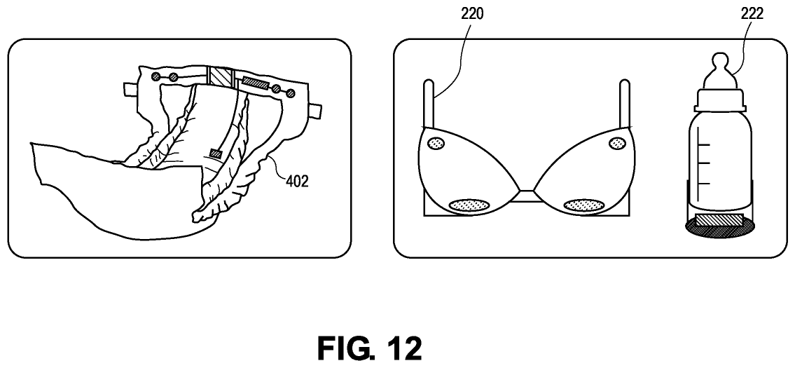

FIG. 12 is a schematic drawing of a system that may be used to perform at least a portion of the method in FIG. 11, according to an embodiment. The system may include the smart bra 220 and/or smart bottle 222 that is/are configured to capture bioimpedance and/or pressure measurements, which may then be used to quantify the amount of breast milk, formula, and/or water consumed the infant. The system may also include the smart diaper 402, which may include one or more sensors that are configured to measure excretions (e.g., urine and/or feces) from the infant. The amount consumed may be compared to the amount excreted. For example, if the amount consumed is less than the amount excreted by a predetermined amount and/or rate, then it may be determined that the infant is dehydrated and/or has diarrhea.

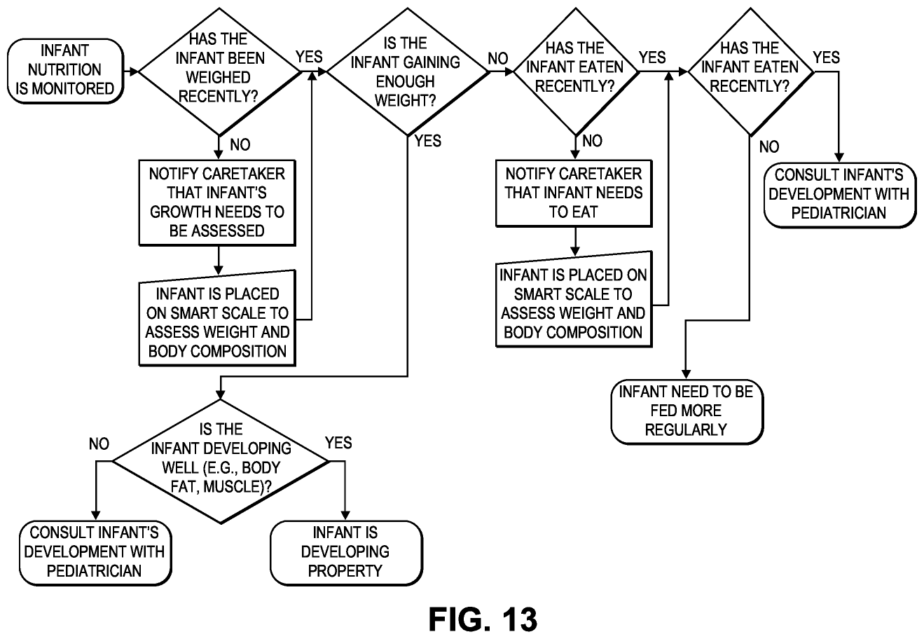

FIG. 13 is a flowchart of a method for monitoring an infant's nutrition, growth, and/or development using one or more sensor devices, according to an embodiment. As shown, the method may be used to determine whether the infant is eating at a predetermined rate and/or whether the infant is developing body fat, muscle, etc. at a predetermined rate. The method may be performed using the system shown in FIG. 9.

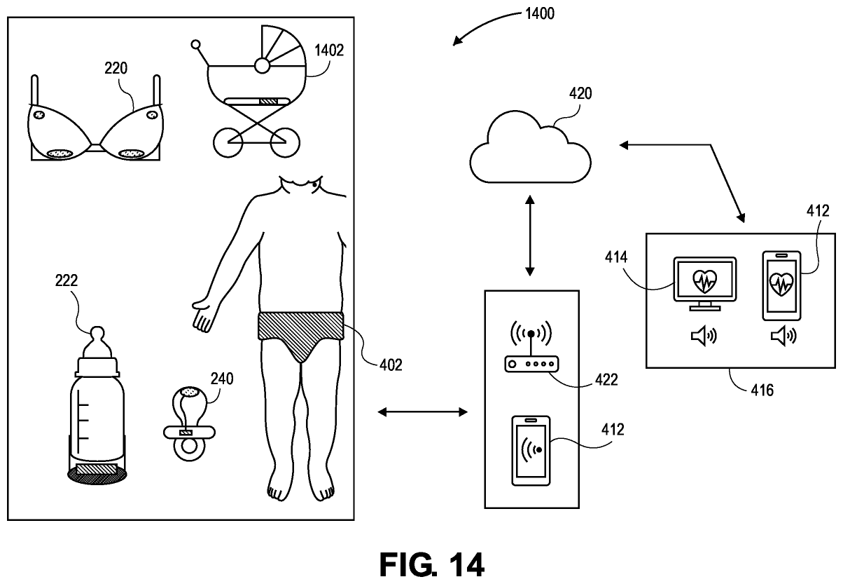

FIG. 14 is a schematic drawing of a wireless connected monitoring system 1400 for monitoring an infant's nutrition, according to an embodiment. The system 1400 may include the diaper 402 to measure infant output/excretion and/or the onset of colic. The system 1400 may also include the smart bra 220 to measure the amount of breast milk consumed by the infant. The system 1400 may include the smart bottle 222 for measuring the amount of fluid that the infant consumes. The system 1400 may also include the smart pacifier 240 to measure temperature and/or saliva composition of the infant. The system 1400 may also include a smart stroller 1402 having one or more sensors to measure weight and/or body composition of the infant. The system 1400 may also include the smartphone 412 to access and display data. The system 1400 may also include the computer 414 to access and display data. The system 1400 may also include the visual and audio data display 416 that is configured to generate and/or display graphs, charts, and/or alarms. The system 1400 may also include the gateway to interface between the aforementioned devices and the cloud data storage 420. The system 1400 may be configured to transmit the data via the router 422 or via Bluetooth, BLE, etc.

### System and Method to Measure Infant Feeding and Output

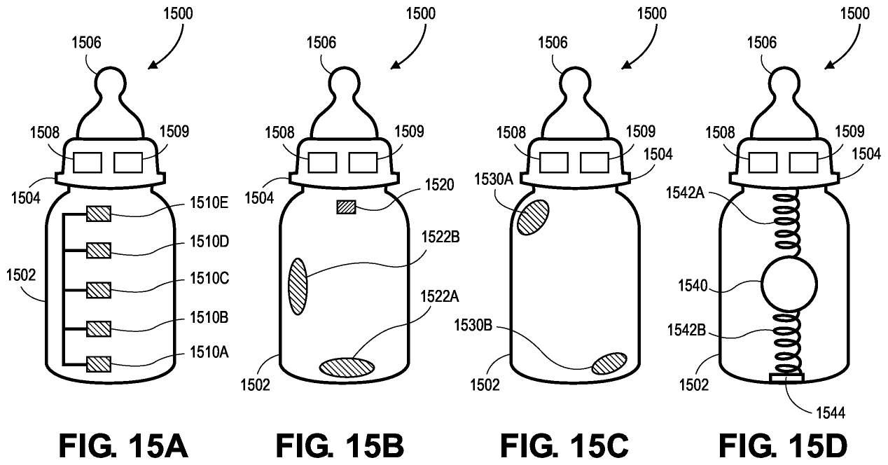

FIGS. 15A-15D illustrate a nursing/feeding bottle 1500 for providing milk or infant formula to an infant, according to an embodiment. The nursing bottle 1500 may include a bottle 1502 that defines an inner volume for receiving/storing the milk or infant formula. The nursing bottle 1500 may also include a cap 1504 that is configured to be coupled to the bottle 1502, and a teat 1506 that extends at least partially through the cap 1504. As described below, the nursing bottle 1500 may also include one or more sensors that are configured to measure one or more parameters. The sensors may be coupled to and/or positioned at least partially within the bottle 1502, the cap 1504, the teat 1506, or a combination thereof.

The nursing bottle 1500 may also include a computing system 1508 that is coupled to and/or positioned at least partially within the bottle 1502, the cap 1504, the teat 1506, or a combination thereof. The computing system 1508 may be configured to receive the one or more parameters and to determine an amount of the milk or infant formula remaining in the bottle 1502, an amount of the milk or infant formula consumed from the bottle 1502 by an infant, or both based at least partially upon the one or more parameters.

In one embodiment, the nursing bottle 1500 may also include a display 1509 that is coupled to and/or positioned at least partially within the bottle 1502, the cap 1504, the teat 1506, or a combination thereof. The display 1509 may be configured to display the amount of the milk or infant formula remaining in the bottle 1502, the amount of the milk or infant formula consumed from the bottle 1502 by an infant, or both. In another embodiment, the computing system 1508 may be configured to (e.g., wirelessly) transmit the one or more parameters to an external device (e.g., a smartphone, tablet, laptop, etc.), which may display the amount of the milk or infant formula remaining in the bottle 1502, the amount of the milk or infant formula consumed from the bottle 1502 by an infant, or both.

FIG. 15A is a schematic side view of the nursing bottle 1500 with photodetectors 1510A-1510E to measure the amount of liquid (e.g., milk or formula) in the bottle 1502. The photodetectors 1510A-1510E may be coupled to an outer and/or inner surface of the bottle 1502 and configured to measure ambient light. The photodetectors 1510A-1510E may be positioned at different heights, corresponding to different volumes in the bottle 1502. For example, the photodetector 1510A may be positioned at a height that corresponds to 5 oz, the photodetector 1510B may be positioned at a height that corresponds to 4 oz, and so on. When the photodetectors 1510A-1510E are covered by milk or formula, they may be dimmer than when uncovered (i.e., they may measure less ambient light). The computing system 1508 may receive the measured light from the photodetectors 1510A-1510E and determine an amount of milk or infant formula remaining in the bottle 1502, an amount of the milk or infant formula consumed from the bottle 1502 by an infant, or both based at least partially upon the measured light.

FIG. 15B is a schematic side view of the nursing bottle 1500 with an accelerometer 1520 and/or pressure sensors 1522A, 1522B coupled thereto and/or positioned therein. The accelerometer 1520 may be configured to measure the orientation of the bottle 1502, and the pressure sensors 1522A, 1522B may be configured to measure the amount of milk or formula in the bottle 1502. In one embodiment, the pressure sensor 1522A may be positioned on a bottom wall of the bottle 1502, and the pressure sensor 1522B may be positioned on a side wall of the bottle 1502. The computing system 1508 may receive the orientation and/or pressure measurements and determine an amount of milk or infant formula remaining in the bottle 1502, an amount of the milk or infant formula consumed from the bottle 1502 by an infant, or both based at least partially upon the orientation and/or pressure measurements.

FIG. 15C is a schematic side view of the nursing bottle 1500 with bioimpedance electrodes 1530A, 1530B that are configured to measure the amount of liquid (e.g., milk or formula) in the bottle 1502. The first electrode 1530A may be positioned above the second electrode 1530B. The electrodes 1530A, 1530B may measure the impedance therebetween, which may depend upon the amount of milk and/or formula in the bottle 1502. The computing system 1508 may receive the impedance measurements and determine an amount of milk or infant formula remaining in the bottle 1502, an amount of the milk or infant formula consumed from the bottle 1502 by an infant, or both based at least partially upon the impedance measurements. An alternative embodiment may include a bioimpedance array, similar to the photodiodes in FIG. 15A.

FIG. 15D is a schematic side view of the nursing bottle 1500 with a buoyant object (e.g., ball) 1540 attached to one or more biasing members (e.g., springs) 1542A, 1542B to measure the amount of milk or formula in the bottle 1502. More particularly, the buoyant ball 1540 may be coupled to and/or positioned between a top spring 1542A and a bottom spring 1542B. The buoyant ball 1540 may be or include a plastic or rubber ball that is full of air. In other embodiments, the buoyant ball 1540 may be made of any material with density lower than milk/formula that can create enough buoyant force to compress and/or expand the springs 1542A, 1542B. The top spring 1542A and/or bottom spring 1542B may be connected to a pressure sensor 1544 made of a piezoelectric material that may capture measurements that are based at least partially upon the pulling force exerted by the spring 1542A and/or 1542B coupled thereto. The computing system 1508 may receive the measurements and determine an amount of milk or infant formula remaining in the bottle 1502, an amount of the milk or infant formula consumed from the bottle 1502 by an infant, or both based at least partially upon the measurements.

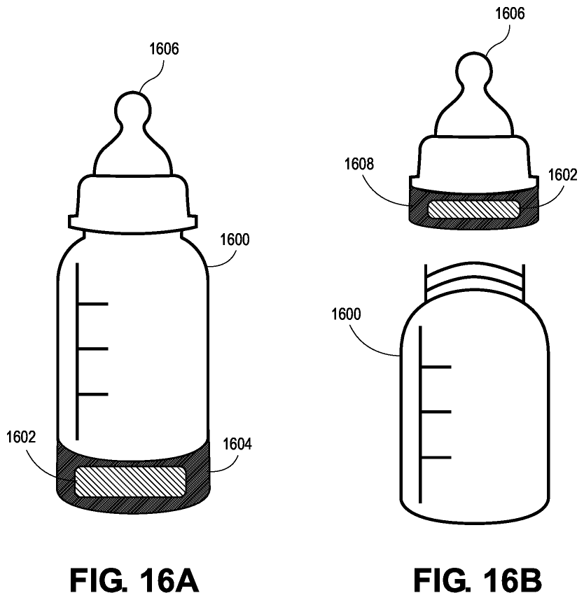

FIG. 16A is a schematic side view of the smart feeding bottle 1600 that measures the amount of milk or formula consumed by the baby where the processing and communications module is in the body of the bottle 1600, according to an embodiment. A processing and communications module 1602 may be positioned in (or coupled to an exterior of) the smart bottle 1600. The module 1602 may be configured to collect sensor data from the sensors in FIGS. 15A-15D and transmit this information wirelessly to a phone, tablet, or modem. A bottle attachment or casing 1604 may be used to contain the module 1602. The attachment or casing 1604 may be connected to the sensors in the bottle 1600. Alternative embodiments may include a breast pump bottle, a feeding bottle cap 1606, and/or breast pump bottle cap or adaptor.

FIG. 16B is a schematic side view of the smart feeding bottle 1600 that measures the amount of milk or formula consumed by the baby where the processing and communications module is in the cap 1608 of the bottle 1600, according to an embodiment.

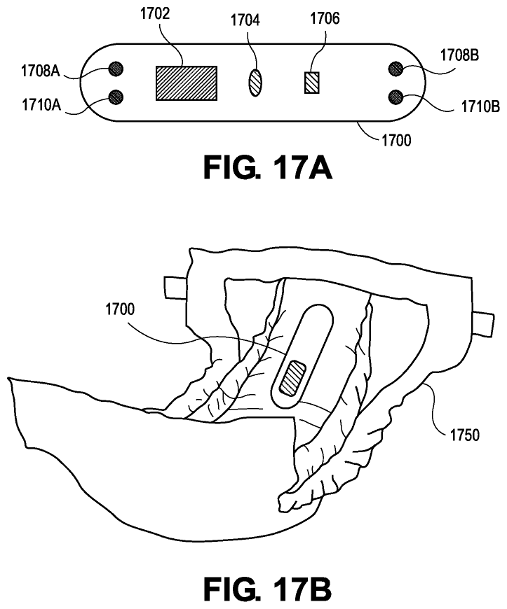

FIG. 17A is a schematic view of a biosensor device (e.g., a patch) 1700, according to an embodiment. The device 1700 may include a module 1702 that is configured to process and communicate measured data, as discussed below. The module 1702 may be powered by a battery. In another embodiment, the module 1702 may be powered biochemically with reagents provided by the infant's urine. In another embodiment, the module 1702 may be or include a non-battery radio-frequency sensor that can be scanned with a smartphone with NFC from outside the diaper. The device 1700 may also include one or more sensors 1704 configured to measure temperature, moisture, pH, biochemical elements, or a combination thereof. The device 1700 may also include a (e.g., disposable) adhesive patch 1706 including biosensors and other components. The device 1700 may also include one or more bioimpedance sense electrodes 1708A, 1708B and one or more bioimpedance drive electrodes 1710A, 1710B. In one embodiment, the sense electrodes 1708A, 1708B may be configured to sense one or more bioimpedance signals that are not in response to the drive electrodes 1710A, 1710B. In another embodiment, the sense electrodes 1708A, 1708B may be configured to sense one or more bioimpedance signals that are in response to the drive electrodes 1710A, 1710B. For example, the drive electrodes 1710A, 1710B may be configured to introduce an electrical current into the skin of the infant.

FIG. 17B is a perspective view of the biosensor device 1700 placed in a diaper 1750, according to an embodiment. The device 1700 may be placed in an area of the diaper 1750 where urination and/or defecation occur.

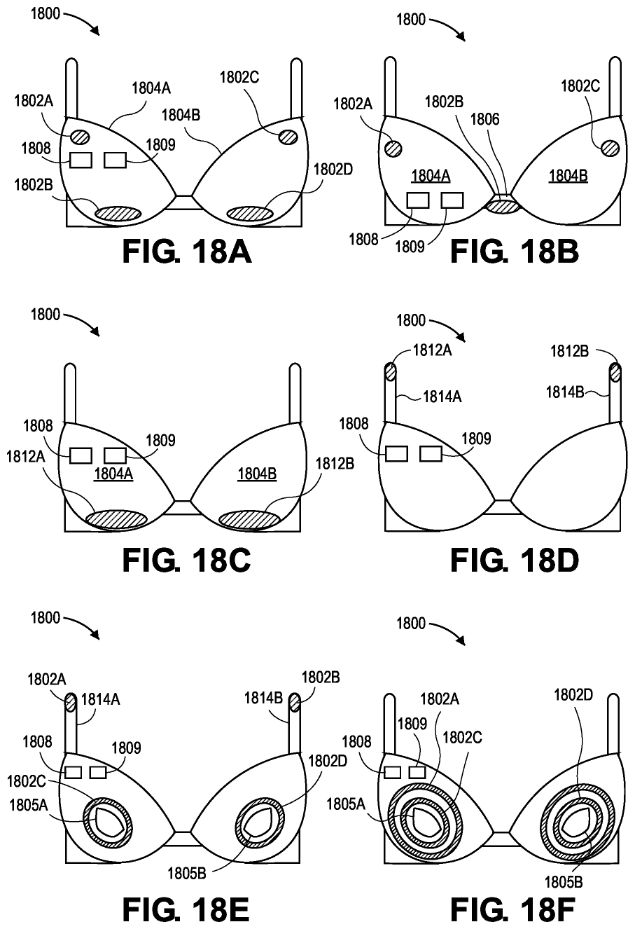

FIGS. 18A-18F illustrate a smart nursing bra 1800 that is configured to measure/determine the amount of milk produced by the mother's breast(s) and/or consumed by the infant during breastfeeding, according to an embodiment. The bra 1800 may include cups 1804A, 1804B with nipple openings 1805A, 1805B. The bra 1800 may also include a center gore 1806 and straps 1814A, 1814B.

As described below, the bra 1800 may also include one or more sensors that are configured to measure one or more parameters. The one or more sensors may be coupled to the cups 1804A, 1804B, the gore 1806, the straps 1814A, 1814B, or a combination thereof. The bra 1800 may also include a computing system 1808 that is coupled to the cups 1804A, 1804B, the gore 1806, the straps 1814A, 1814B, or a combination thereof. The computing system 1808 may be configured to receive the one or more parameters and to determine the amount of milk produced by the mother's breast(s) and/or consumed by the infant during breastfeeding based at least partially upon the one or more parameters.

In one embodiment, the bra 1800 may also include a display 1809 that is coupled to the cups 1804A, 1804B, the gore 1806, the straps 1814A, 1814B, or a combination thereof. The display 1809 may be configured to display the amount of milk produced by the mother's breast(s) and/or consumed by the infant during breastfeeding. In another embodiment, the computing system 1808 may be configured to (e.g., wirelessly) transmit the one or more parameters to an external device (e.g., a smartphone, tablet, laptop, etc.), which may display the amount of milk produced by the mother's breast(s) and/or consumed by the infant during breastfeeding.

FIG. 18A is a front view of the bra 1800 that measures the amount of milk delivered during breastfeeding using bioimpedance, according to an embodiment. The bra 1800 may include one or more bioimpedance electrodes 1802A-1802D. The bioimpedance electrodes 1802A-1802D may be coupled to and/or positioned at least partially within the cups 1804A, 1804B and configured to contact the skin. In this embodiment, the bioimpedance electrodes 1802A-1802D may be located on the top and/or bottom of each cup 1804A, 1804B.

The bioimpedance electrodes 1802A-1802D may be configured to measure the bioimpedance (e.g., a variation in the bioimpedance) of the breasts during breastfeeding. The bioimpedance may vary in response to the amount of milk that is generated by and/or flows out of the breasts. Thus, the amount of milk that flows out of the breasts may be determined based at least partially upon the measured variation in the bioimpedance.

The computing system 1808 may receive the bioimpedance measurements from the bioimpedance electrodes 1802A-1802D and determine the amount of milk produced by the mother's breast(s) and/or consumed by the infant during breastfeeding based at least partially upon the bioimpedance measurements.

FIG. 18B is a front view of the smart bra 1800 that measures the amount of milk delivered during breastfeeding using bioimpedance, according to an embodiment. The bioimpedance electrodes 1802A-1802C may be located on the cup 1804A, the cup 1804B, and the center gore 1806 between the cups 1804A, 1804B.

FIG. 18C is a front view of the smart bra 1800 that measures the amount of milk delivered during breastfeeding using pressure sensors 1812A, 1812B in the cups 1804A, 1804B or underwire, according to an embodiment. More particularly, the pressure sensors 1812A, 1812B may be located on the bottom of the cups 1804A, 1804B. The pressure sensors 1812A, 1812B may be configured to measure a pressure (e.g., a variation in the pressure) in the breasts. The pressure may vary in response to the amount of milk that is produced by and/or flows out of the breasts.

The computing system 1808 may receive the pressure measurements from the pressure sensors 1812A, 1812B and determine the amount of milk produced by the mother's breast(s) and/or consumed by the infant during breastfeeding based at least partially upon the pressure measurements.

FIG. 18D is a front view of the smart bra 1800 that measures the amount of milk delivered during breastfeeding using pressure sensors 1812A, 1812B in the straps 1814A, 1814B, according to an embodiment.

FIG. 18E is a front view of the smart bra 1800 that measures the amount of milk delivered during breastfeeding using bioimpedance sensors 1802A-1802D, according to an embodiment. The sensor 1802A is located on one strap 1814A, and the sensor 1804B is located on the other strap 1814B. The sensor 1802C is located proximate to (e.g., at least partially around) the nipple opening 1805A in the cup 1804A, and the sensor 1802D is located proximate to (e.g., at least partially around) the nipple opening 1805B in the cup 1804B.

FIG. 18F is a front view of a smart nursing bra 1800 that measures the amount of milk delivered during breastfeeding using bioimpedance sensors 1802A-1802D, according to an embodiment. Outer bioimpedance electrodes 1802A, 1802B may be located at least partially around the nipple openings 1805A, 1805B. Inner bioimpedance electrodes 1802C, 1802D may also be positioned at least partially around the nipple openings 1805A, 1805B. The inner bioimpedance electrodes 1802C, 1802D may be positioned at least partially between the outer bioimpedance electrodes 1802A, 1802B and the nipple openings 1805A, 1805B.

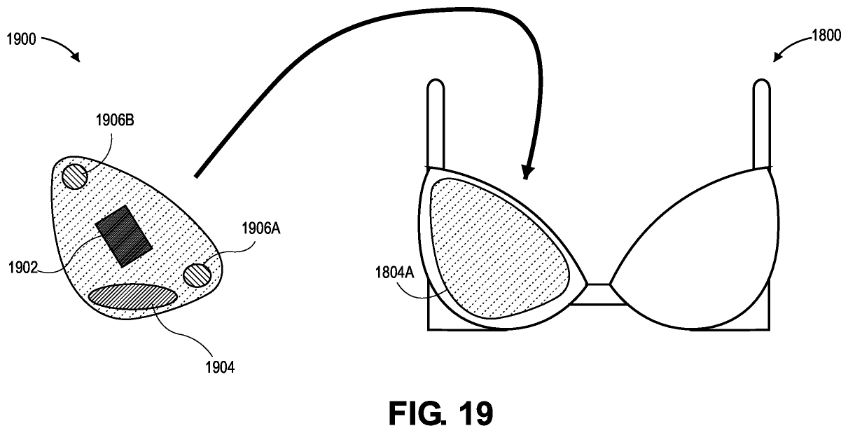

FIG. 19 is a schematic view of a smart pad insert 1900 that measures the amount of milk delivered during breastfeeding, according to an embodiment. The insert 1900 may be positioned at least partially within the bra 1800 (e.g., within one of the cups 1804A, 1804B). For example, the insert 1900 may be positioned between an inner layer and an outer layer of the cup 1804A. The insert 1900 may include sensors and electronics. More particularly, the insert 1900 may include a chip (e.g., a processing unit) 1902, which may be connected to or include a wireless communications module, an accelerometer, and/or a battery). The insert 1900 may also include pressure sensor 1904 located at the bottom of the insert 1900 that is configured to measure the pressure variation in the breast while the infant is breastfeeding. The insert 1900 may also a bioimpedance electrode 1906A located on a side of the insert 1900, which enters in contact with the skin when placed inside the bra 1800. The insert 1900 may also include another bioimpedance electrode 1906B located on the other side of the insert 1900, which enters in contact with the skin when placed inside the bra 1800. The sensors 1906A, 1906B may measure the bioimpedance variation in the breast while the infant is breastfeeding.

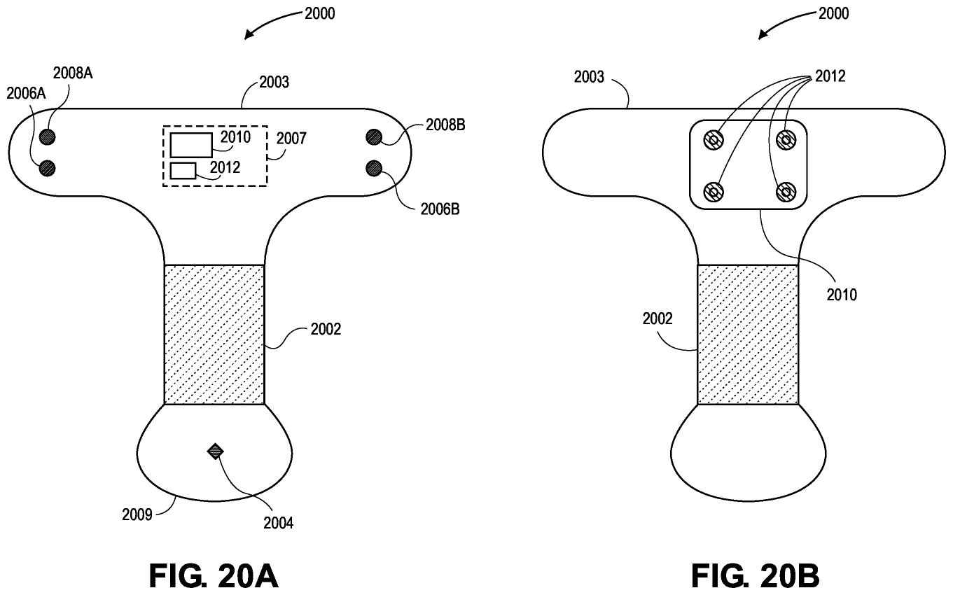

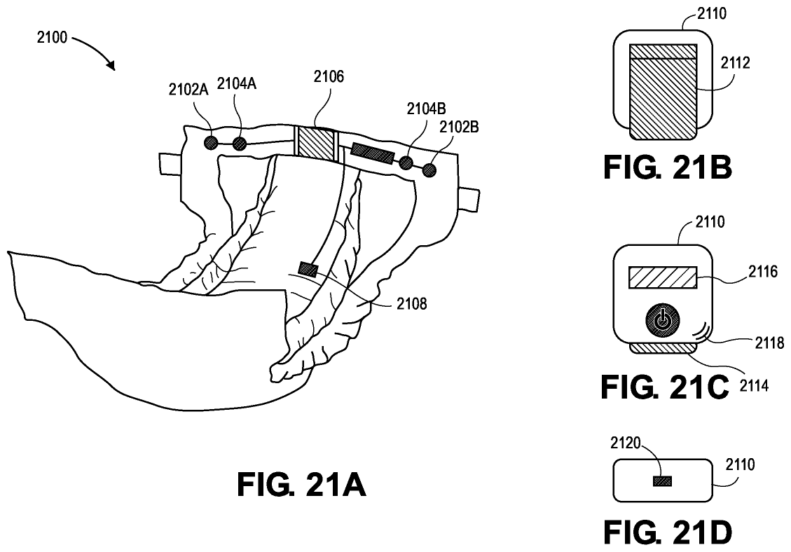

FIG. 20A is a front view (facing towards the body) of a wearable sensor insert 2000 worn within a diaper or onesie, according to an embodiment. In an embodiment, the insert 2000 may be configured to be placed into a diaper (e.g., component 2100 in FIG. 21A) worn by an infant. The insert 2000 may include an embedded electronic circuit 2007 that controls measurements characterizing the infant's physiology and detecting whether the infant has urinated or defecated. The insert 2000 may be or include a soft, flexible (e.g., rubber) material. The insert 2000 may include an upper region 2003, a lower region 2009, and a flexible connector 2002 therebetween.

The electronic circuit 2007 may include, among other components, an impedance circuit component 2010 that generates two sources of high-frequency (e.g., 5-500 kHz), low-amperage (e.g., 0.01-10 mA) electrical current that are separately injected into an area to be measured. The electrical currents may be about 180° out of phase with each other, and are referred to herein as ‘outputs’ from the impedance circuit component 2010. Additionally, the impedance circuit component 2010 receives signals measured from the area that are affected by the current, referred to herein as ‘inputs.’ The areas for measurement, for example, are the infant's chest and/or belly (e.g., for physiological measurements), and a region proximal to the infant's genitalia (e.g., for determining if the infant has urinated or defecated).

The impedance circuit component 2010 may further include a differential amplifier that receives the inputs, which are generated by separate electrodes in contact with the areas as described in more detail below. The differential amplifier differentiates the inputs to generate a voltage, and then amplifies the voltage to a level that is digitized and ultimately processed by an algorithm (e.g., one running on a microprocessor within the electronic circuit 2007) to determine a specific measurement corresponding to the infant. The electronic circuit 2007 additionally includes a multiplex component 2012 that ‘switches’ inputs and outputs to separate sensors located in the different areas, as described in more detail below, to sequentially measure the infant's physiology and determine if they have urinated or defecated.

More specifically, as shown in FIG. 20A, in embodiments, the upper region 2003 includes a pair of ‘drive’ electrodes 2006A, 2006B located on the region's distal ends that each receive separate current inputs from the impedance circuit component 2010 when the multiplex component 2012 is switched thereto. Complementing these is a pair of ‘sense’ electrodes 2008A, 2008B, each located proximal to a corresponding drive electrode 2006A, 2006B, that receive individual bio-electrical signals from the infant, and port these signals to the impedance circuit component 2010 for processing. The electrodes may be or include materials that are soft, flexible, and conductive (e.g., a conductive elastomer or fabric) that contact the infant's belly when the insert 2000 is placed within the diaper. The drive electrodes 2006A, 2006B and sense electrodes 2008A, 2008B may be spaced apart as much as possible in the upper region 2003, as this increases the difference in the bio-electrical signals that the impedance circuit component 2010 ultimately measures, which in turn maximizes the level of the final voltage the impedance circuit component 2010 generates.

During a measurement, the impedance circuit component 2010 generates time-dependent AC and DC waveforms from the infant. For physiological measurements, these are processed, as described in detail below, to yield parameters such as respiration rate, heart rate, stroke volume, cardiac output, and/or fluid levels associated with the infant.

Periodically or episodically, the multiplex component 2012 switches the inputs and outputs to a separate ‘waste’ sensor 2004 located in the lower region 2009 of the insert 2000. The waste sensor 2004 also include pairs of sense and drive electrodes (not shown), with a first waste electrode featuring a first sense electrode and first drive electrode electrically shorted together, and a second waste electrode featuring a second sense electrode and second drive electrode electrically shorted together. Here, the first and second waste electrodes may be or include conductive materials (e.g., rubber or fabric, like those used for physiological measurements; alternatively conductive metal or plastic materials) and are located relatively close together compared to the electrodes in the upper region 2003. Urine and feces excreted by the infant features different electrical conductivity, and thus generates unique voltages when measured by the impedance circuit component 2010. During a measurement, the microprocessor receives these voltages and processes them with an algorithm (e.g., one featuring a look-up table) to determine if the infant has urinated or defecated.

The multiplex component 2012 switches the impedance circuit component 2010 to primarily measure physiological signals from the infant using the drive 2006A, 2006B and sense 2008A, 2008B electrodes, and only occasionally switches to the waste sensor 2004 for a relatively short measurement (e.g., just a few seconds) indicating if urine or feces is present. Wires (not shown) that connect the circuit component 2007 to the electrodes 2006A, 2006B, 2008A, 2008B and waste sensor 2004 may be embedded within the insert.

FIG. 20B is a back view (facing away from the body) of the wearable sensor insert 2000 worn inside a diaper or onesie, according to an embodiment. The insert 2000 may include a housing 2010, which may include a microcontroller, an accelerometer, a wireless communication module, and/or a battery. The insert 2000 may also include one or more magnets 2012 for a charging port attachment. The magnets 2012 may also serve as an attachment for a detachable case.

FIG. 21A is a perspective view of a disposable electronic smart diaper 2100, according to an embodiment. The diaper 2100 may include one or more drive electrodes 2102A, 2102B and one or more sense electrodes 2104A, 2104B. The diaper 2100 may also include a clipping area 2106. In another embodiment, instead of or in addition to the clipping area 2106, the diaper 2100 may include conductive ink, tape, and/or Velcro. The diaper 2100 may be configured to exchange power and/or information between sensors in diaper 2100 and an attachment. The diaper 2100 may also include a sensor 2108 to detect urination and/or defecation by detecting changes in one or more of color, moisture, temperature, biochemical molecules, conductivity, or a combination thereof. The measurements from the electrodes 2102A, 2102B, 2104A, 2104B across the abdomen may be combined with the measurements from the sensor 2108 to determine the volume of liquid discharged from the bladder during urination.

FIG. 21B is a back view of a reusable case 2110 clipped to the disposable electronic diaper 2100, according to an embodiment. The reusable case 2110 may include a processing and communications module. The reusable case 2110 may also include an accelerometer and/or gyroscope to measure the position, activity, and/or orientation of the infant. The case 2110 may include a clip 2112 for attachment to the electronic diaper 2100.

FIG. 21C is a front view of the reusable case 2110 clipped to the disposable electronic diaper 2100, according to an embodiment. The case 2110 may include a button input 2114 to program the processing and communications module and/or accelerometer. The case 2110 may also include a visual display screen 2116. The case 2110 may also include an audio module 2118 with speaker output and microphone input (e.g., to detect crying).

FIG. 21D is a bottom view of the reusable case 2110 clipped to the disposable electronic diaper 2100, according to an embodiment. The case 2110 may include a charging port (e.g., USB-C) 2120.

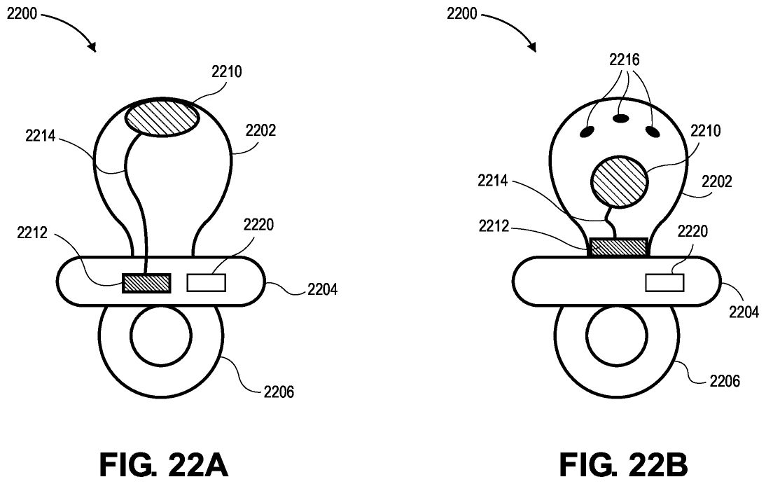

FIG. 22A is a side view of a smart pacifier 2200 with one or more sensors 2210 for measuring nutrition-related information from saliva and temperature, according to an embodiment. The pacifier 2200 may include a teat (also referred to as a nipple) 2202, a mouthpiece 2204, and a handle 2206. The sensors 2210 may be coupled to and/or positioned at least partially within the teat 2202 and configured to measure the temperature of the infant. The sensors 2210 may also or instead be configured to measure biochemical data. For example, the sensors 2210 may be configured to measure bilirubin, pH, enzymes, glucose, or a combination thereof, without the need to prick the infant.

The pacifier 2200 may also include a computing system (e.g., a processing and communications module) 2212 that is coupled to and/or positioned at least partially within the teat 2202, the mouthpiece 2204, and/or the handle 2206. The computing system 2212 may be configured to receive the measured data from the sensors 2210. The computing system 2212 may be configured determine the temperature of the infant based at least partially upon the temperature measurements. The computing system 2212 may also or instead be configured to determine the bilirubin, pH, enzymes, glucose, or a combination thereof based at least partially upon the measured biochemical data.

In one embodiment, the pacifier 2200 may also include a display 2220 that is coupled to the teat 2202, the mouthpiece 2204, and/or the handle 2206. The display 2220 may be configured to display the temperature and/or biochemical data. In another embodiment, the computing system 1808 may be configured to (e.g., wirelessly) transmit the one or more parameters to an external device (e.g., a smartphone, tablet, laptop, etc.), which may display the temperature and/or biochemical data.

FIG. 22B is a side view of the smart pacifier 2200 with the sensors 2210 inside the teat 2202 for measuring nutrition-related information from saliva and temperature, according to an embodiment. The teat 2202 may include one or more openings 2216 through which the infant's saliva may pass to the sensors 2210.

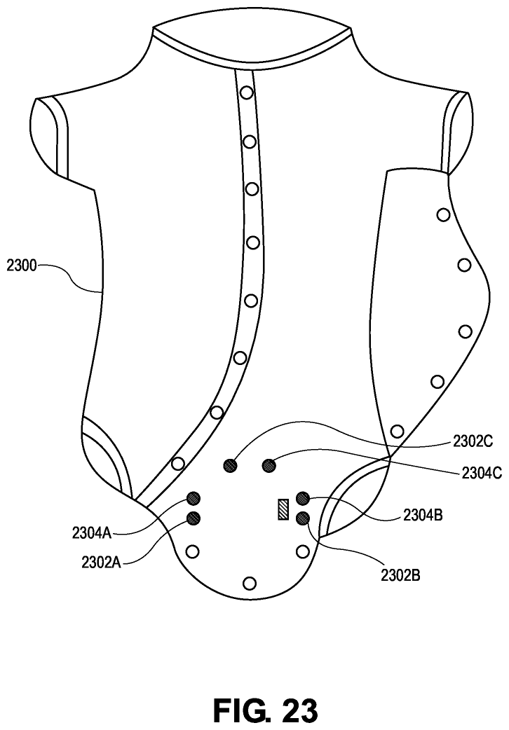

FIG. 23 is a front view of a smart monitoring onesie 2300, according to an embodiment. The onesie 2300 may include a one or more bioimpedance electrodes. More particularly, the onesie 2300 may include one or more bioimpedance sense electrodes 2302A-2302C and one or more bioimpedance drive electrodes 2304A-2304C. The sense electrodes 2302A-2302C may be positioned on the left and right sides of the abdomen and above the abdomen. Similarly, the drive electrodes 2304A-2304C may be positioned on the left and right sides of the abdomen and above the abdomen. The drive electrodes 2304A-2304C may be configured to inject an electrical current into the skin of the infant. The sense electrodes 2302A-2302C may be configured to measure one or more first signals that are not in response to the electrical current. The sense electrodes 2302A-2302C may also or instead be configured to measure one or more second signals that are in response to the electrical current. Thus, the electrodes 2302A-2302C and/or 2302A-2302C may be configured to capture bioimpedance measurements across the abdomen, which may be used to determine the volume of liquid discharged from the bladder during urination and/or the presence of intestinal gas (e.g., colic).

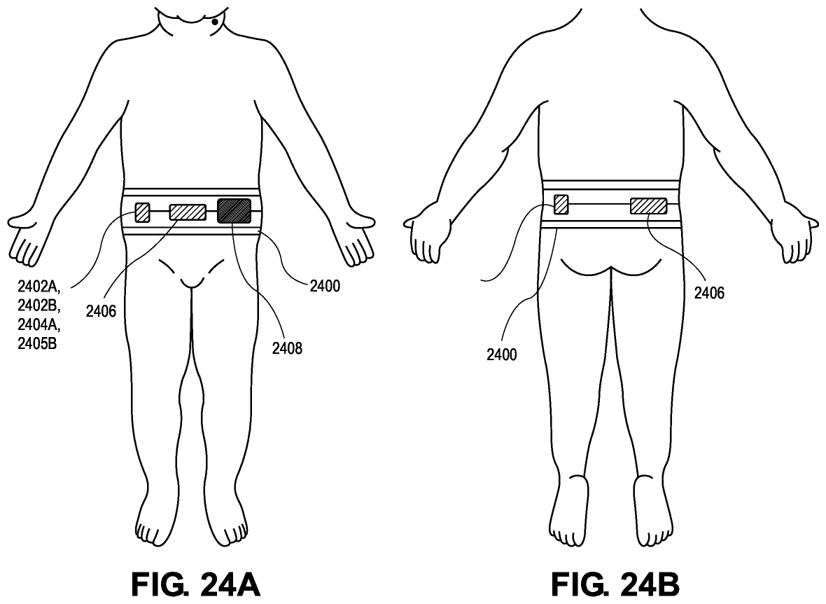

FIG. 24A is a front view of a flexible, stretchable, smart waistband 2400 worn by an infant, and FIG. 24B is a back view of the flexible, stretchable, smart waistband 2400 worn by the infant, according to an embodiment. In another embodiment, the waistband 2400 may be or include a belt or a diaper band. The waistband 2400 may include one or more sense electrodes 2402A, 2402B and one or more drive electrodes 2404A, 2404B. The waistband 2400 may also include a stretch and pressure sensor 2406 configured to measure circumference length and/or tension. The waistband 2400 may also include a processing and communications module 2408 that may include a circuit board, an accelerometer, and/or a gyroscope.

The smart waistband 2400 with bioimpedance and stretch sensors can measure abdominal distension and/or gas accumulation, which may be used to detect colic. This diagnosis may be conveyed wirelessly to the caretaker. For example, increased bloating and gases in the infant may cause the bioimpedance signal to increase (e.g., increased impedance across the abdomen). The increased bloating and gases may also cause the circumference length of the waistband 2400 to increase (e.g., increased abdominal diameter). Additionally, the breathing rate can be picked up with a breath-picking algorithm applied to the accelerometer, bioimpedance, and/or stretch signal to detect the infant's breathing rate. The pressure sensors 2406 can be used with the breathing signal to measure abdominal tension, by factoring the force exerted in the abdomen during breathing and/or the tension in the waistband 2400 at a given diameter/length.

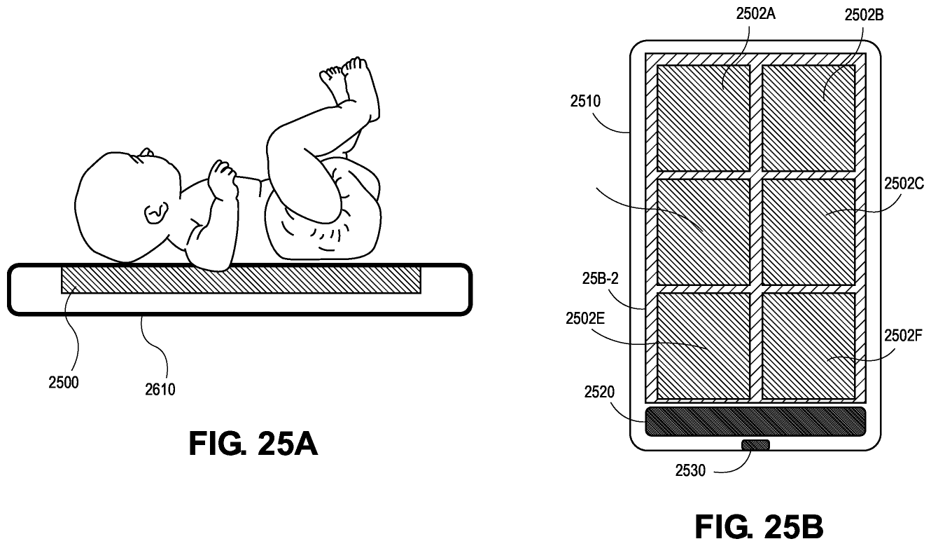

FIG. 25A is a side view of a weight and bioimpedance scale 2500 positioned at least partially on and/or in a changing pad 2510 that can measure weight and body composition in an infant, according to an embodiment. FIG. 25B is a top cross-sectional view of the weight and bioimpedance scale 2500, according to an embodiment. The scale 2500 may include one or more (e.g., six) sections 2502A-2502F that are arranged in a grid-like manner to measure the weight and/or bioimpedance distribution of the infant. These measurements may be used to assess the infant's body composition. The scale 2500 may also include a processing and communications module 2520 that is configured to receive and process the measurements. The module 2520 may determine the weight and/or bioimpedance distribution. The module 2520 may also or instead determine the body composition. The scale 2500 may also include a charging and/or communications port (e.g., USB-C) 2530.

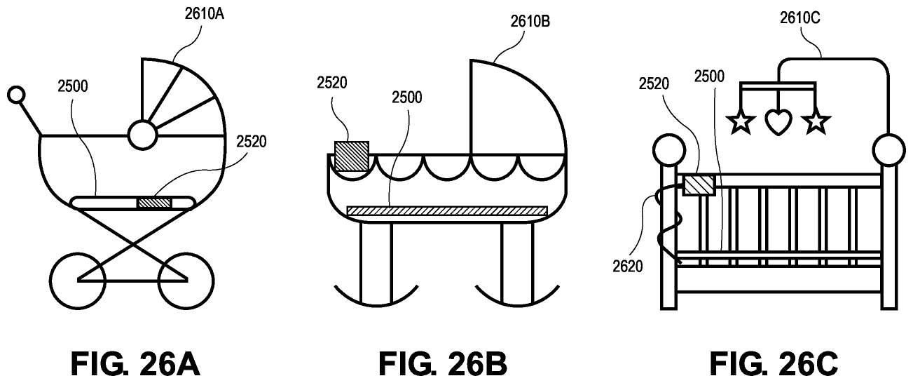

FIG. 26A is a side view of the weight and bioimpedance scale 2500 positioned at least partially on and/or in a stroller 2610A that can measure weight and body composition in an infant, according to an embodiment. FIG. 26B is a side view of the weight and bioimpedance scale 2500 embodied in a bassinet 2610B that can measure weight and body composition in an infant, according to an embodiment. FIG. 26C is a side view of the weight and bioimpedance scale embodied in crib 2610C that can measure weight and body composition in an infant, according to an embodiment. A cable 2620 may connect the 2520 module to the scale 2500.

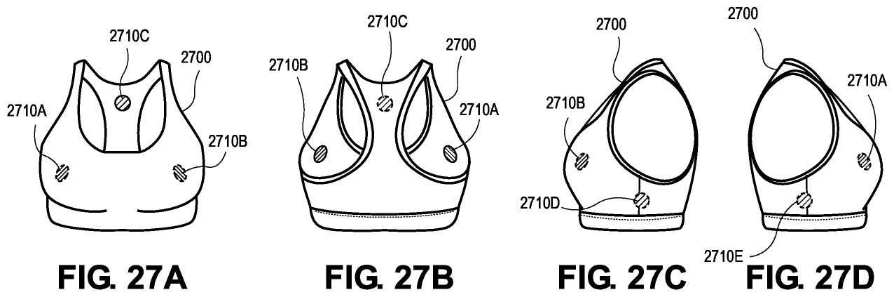

FIGS. 27A-27D illustrate a smart bra 2700 that measures the amount of milk delivered during breastfeeding using bioimpedance, according to an embodiment. The bra 2700 may include one or more bioimpedance electrodes 2710A-2710E. A first electrode 2710A may be coupled to the right cup. A second electrode 2710B may be coupled to the left cup. A third electrode 2710C may be coupled to the back strap. A fourth electrode 2710D may be coupled to the left band or wings. An electrode 2710E may be coupled to the right band or wings. The electrodes 2710A-2710E may be configured to contact the skin of the breasts and to measure the bioimpedance of the skin and/or breasts. The bioimpedance may vary based at least partially upon the amount of milk that flows out of the breasts. For example, a first bioimpedance measurement may be captured before breastfeeding or pumping, and a second bioimpedance measurement may be captured after breastfeeding or pumping. The amount of milk that is produced may be determined based at least partially upon the difference in the measurements.

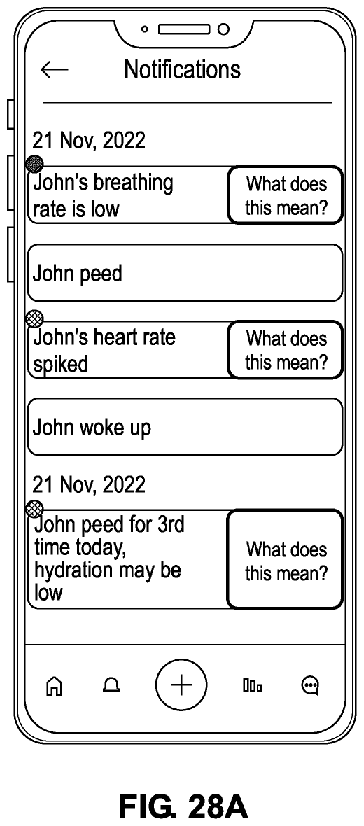

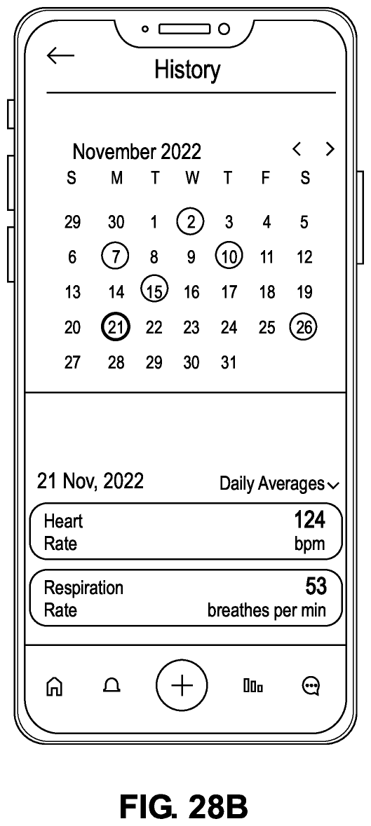

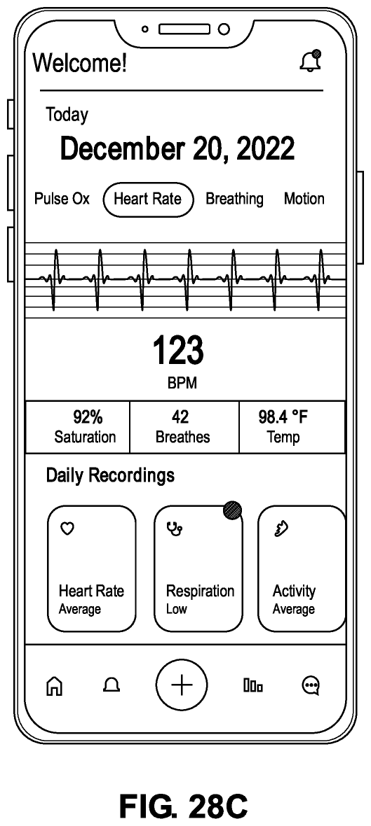

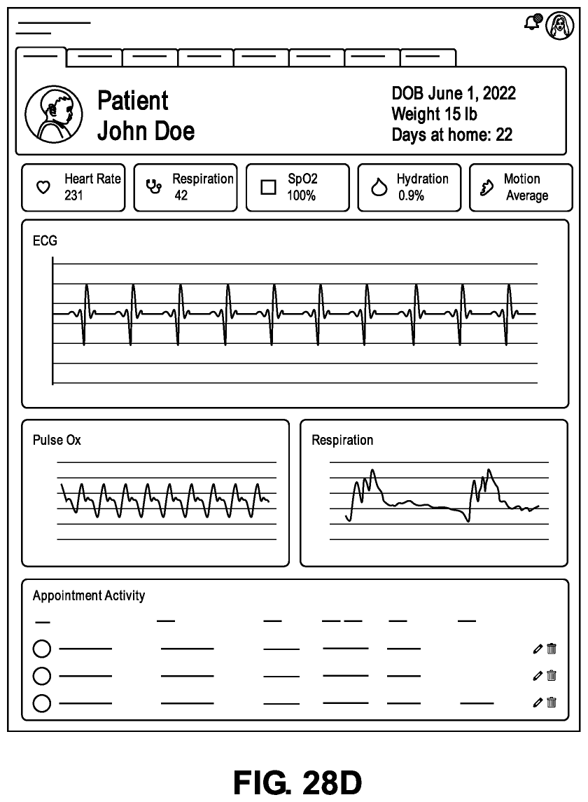

FIGS. 28A-28D are examples of displays (e.g., dashboard views) that may be generated by one or more of the devices herein (e.g., the wearable sensor insert 2000). For example, the displays may be generated by the electronic circuit 2007, the impedance circuit component 2010, the multiplex component 2012, the processing system, or a combination thereof. The displays may show patient information that may be measured or determined (e.g., by the insert 2000). The patient information may be or include the heart rate, respiration, SpO2, hydration, motion, pulse ox, temperature, RR, or a combination thereof.

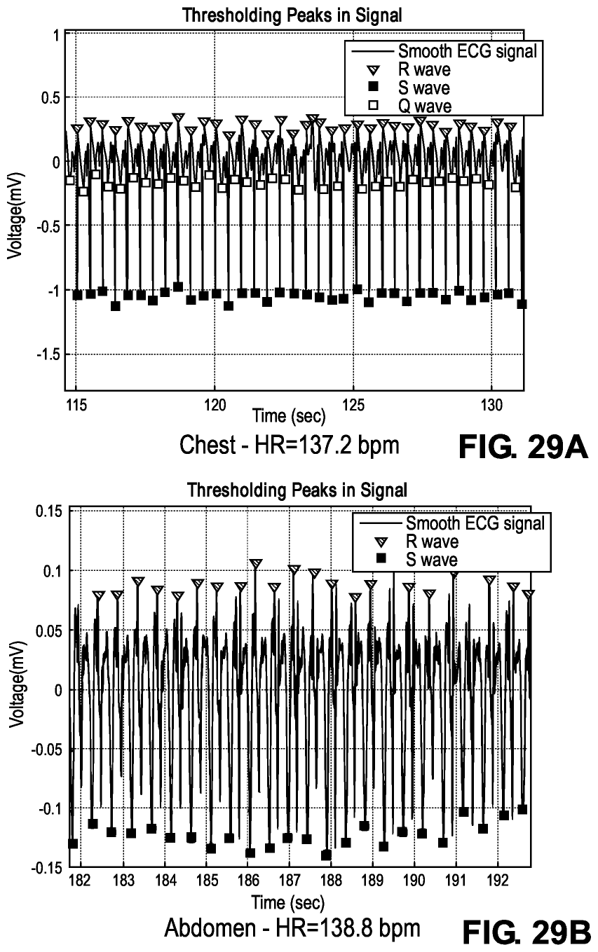

FIG. 29A is a graph showing voltage versus time for the chest. FIG. 29B is a graph showing voltage versus time for the abdomen. As may be seen, the graphs may include the smooth ECG signal, the R wave, the S wave, the Q wave, or a combination thereof.

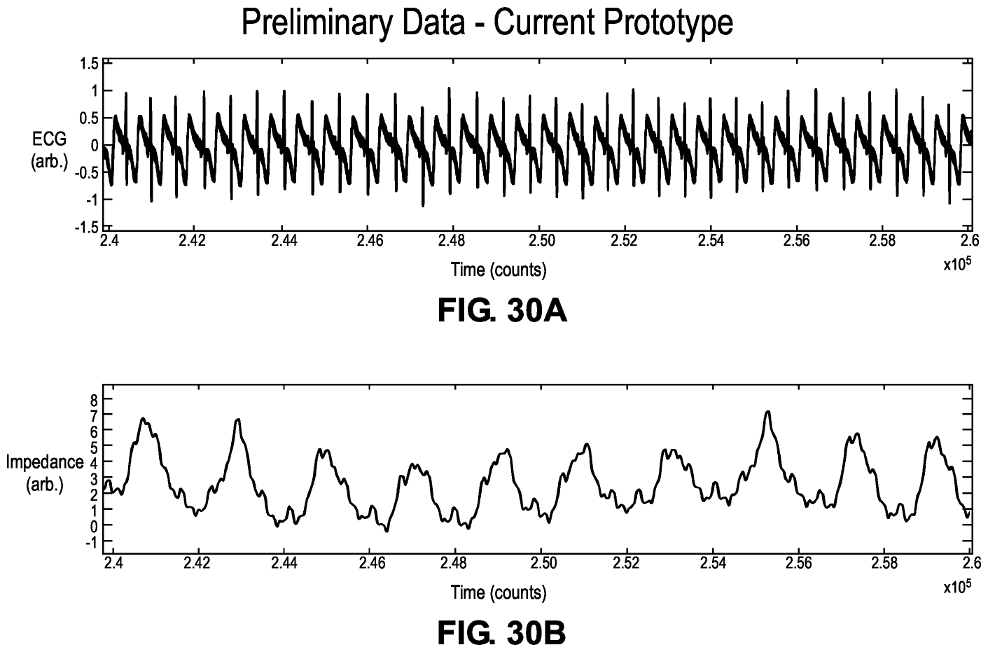

FIG. 30A is a graph showing ECG versus time. FIG. 30B is a graph showing impedance versus time.

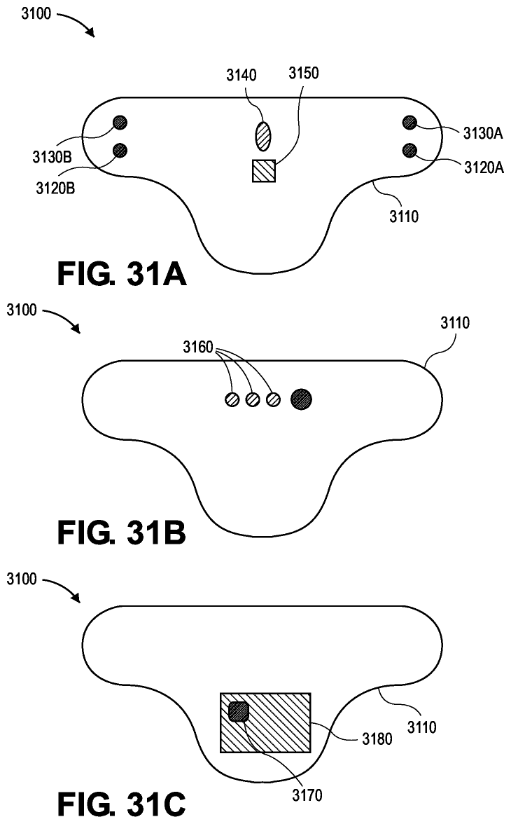

FIG. 31A is a schematic drawing of a front side (facing towards the body) of a wearable sensor insert 3100 worn within a diaper or onesie, FIG. 31B is a schematic drawing of a back side (facing away from the body) of the wearable sensor insert 3100 worn inside a diaper or onesie, and FIG. 31C is a schematic drawing of an inside of a casing 3110 of the wearable sensor insert 3100 worn inside a diaper or onesie, according to an embodiment. The wearable sensor insert 3100 may include first and second drive electrodes 3120A, 3120B and first and second sense electrodes 3130A, 3130B. The wearable sensor insert 3100 may also include an optical sensor 3140, which may be or include a red, a near-infrared LED emitter, and a photodetector. The wearable sensor insert 3100 may also include a temperature sensor 3150. The wearable sensor insert 3100 may also include LED indicators 1360 and an accelerometer 3170. The wearable sensor insert 3100 may also include a computing system (e.g., printed circuit board, controller, battery, communication module) 3180.

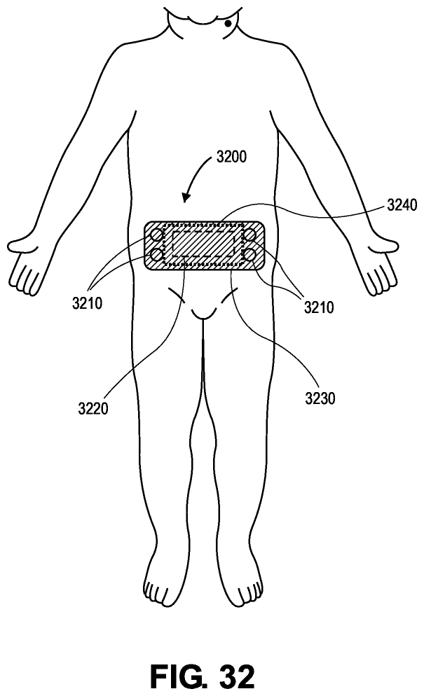

FIG. 32 illustrates a schematic view of an infant wearing a wearable sensor 3200 on the abdomen/belly, according to an embodiment. The wearable sensor 3200 may include one or more electrodes 3210. The wearable sensor 3200 may also include an optical sensor 3220. The wearable sensor 3200 may also include a computing system (e.g., microcontroller and communication modules) 3230. The wearable sensor 3200 may also include circuitry, a battery, and a magnetic clip 3240 that connects through the diaper to the sensor sitting on the abdomen, securing the device in place.

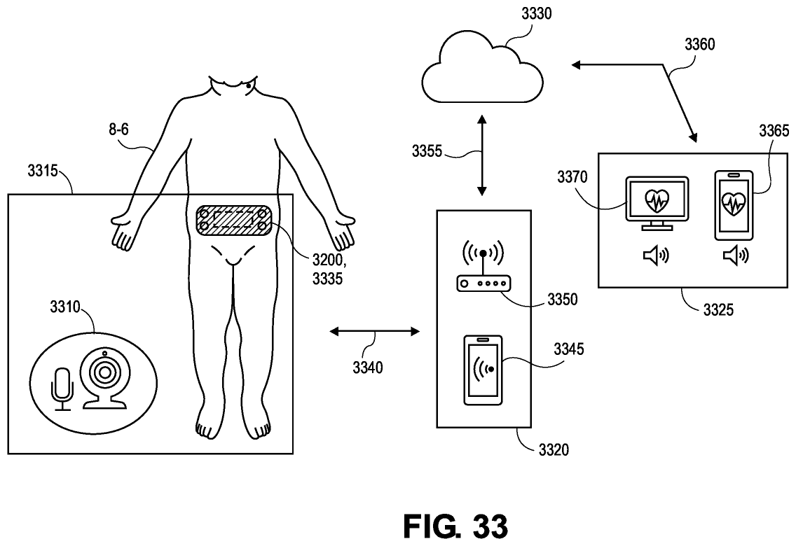

FIG. 33 illustrates a schematic view of a wireless, connected monitoring system 3300 for a patient (e.g., infant) 3305 that integrates to a patient monitoring system, according to an embodiment. The system 3300 may include a microphone and camera 3310 for crib-side monitoring. The system 3300 may also include one or more sensors 3315 to generate data from the patient. The system 3300 may also include a gateway 3320 to interface between the sensors and (e.g., cloud) storage. The system 3300 may also include a visual and/or audio display 3325 that may share graphs, charts, and alarms. Data 3330 in the cloud may be stored and/or processed. The system 3300 may also include a wearable sensor 3335 (which may be the same as the sensor 3200). Data 3340 may be wirelessly transmitted between the sensors and the gateway. The system 3300 may also include a smartphone 3345 and a Wi-Fi router 3350. Data 3355 may be transmitted between the gateway and cloud. Data 3360 may also be transmitted between the cloud display. The system 3300 may also include another smartphone 3365 to access and display the data. The system 3300 may also include a computing system 3370 to access and display the data.

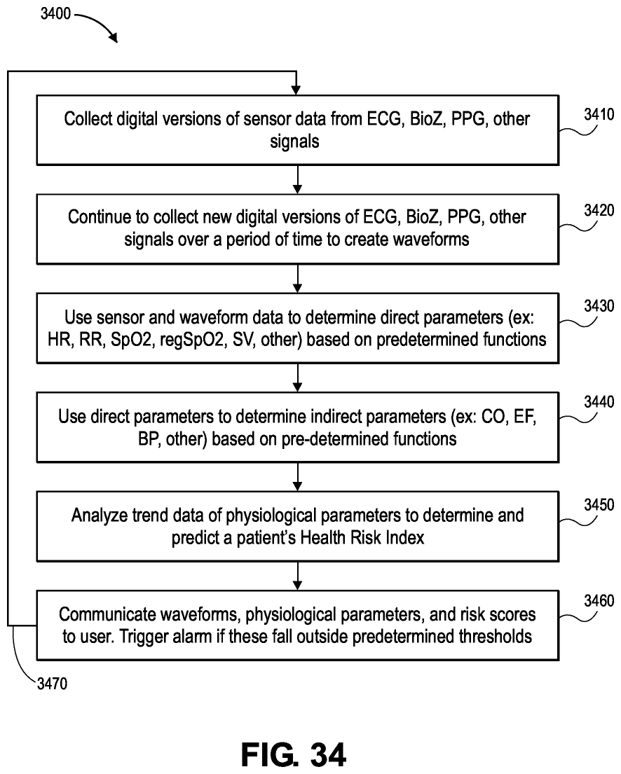

FIG. 34 illustrates a flowchart of a method 3400 for monitoring a patient, according to an embodiment. The method 3400 may include collecting digitally-converted data from sensor module, as at 3410. The method 3400 may also include at further time stamps, continue to collect digitally-converted data from sensor module for a predetermined period of time to generate waveforms of the monitored parameters, as at 3420. The method 3400 may also include using digital sensor data and waveform data to determine direct physiological parameters (e.g., HR, RR, SpO2, regSpO2, SV, other) based on predetermined functions, as at 3430. In an alternative embodiment, the method 3400 can extract more complex biomarkers from these waveforms using pre-determined artificial intelligence algorithms. The method 3400 may also include using digital sensor data, waveform data, determined physiological parameters, and waveform features to determine indirect physiological parameters (e.g., CO, EF, BP, other) based on pre-determined functions, as at 3440. The method 3400 may also include using pre-determined functions to analyze trends of physiological parameters to determine a patient's Health Risk Index based on the patient's current status (e.g., patient not breathing, heart stopped), as at 3450. This may also or instead include analyzing trends of physiological parameters and Health Risk Index to predict a patient's future deterioration (e.g., heart failure, lowering oxygen, increase in temperature). The method 3400 may also include communicating suitable digital data waveforms, determined physiological parameters, and health risk scores to user, as at 3460. This may also or instead include triggering an alarm if these variables fall outside predetermined thresholds. The method 3400 may also include continuing process of data collection, processing, and variable display until the system is turned off, as at 3470.

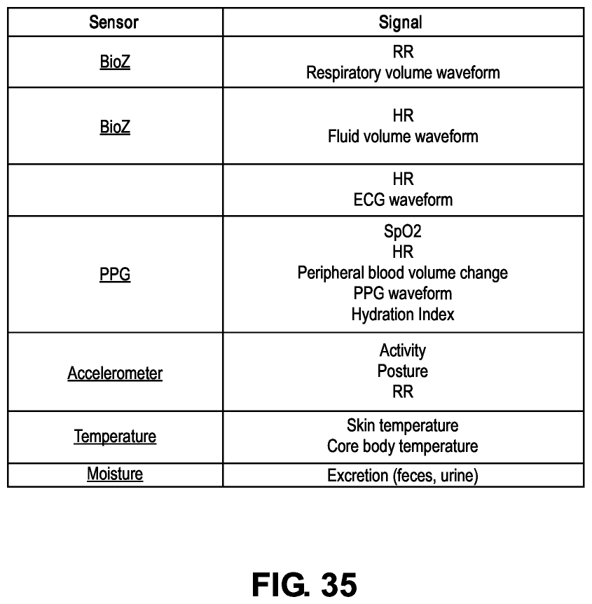

FIG. 35 illustrates a table listing monitoring methods in the system with associated physiological signals.

As used herein, the terms “inner” and “outer”; “up” and “down”; “upper” and “lower”; “upward” and “downward”; “upstream” and “downstream”; “above” and “below”; “inward” and “outward”; and other like terms as used herein refer to relative positions to one another and are not intended to denote a particular direction or spatial orientation. The terms “couple,” “coupled,” “connect,” “connection,” “connected,” “in connection with,” and “connecting” refer to “in direct connection with” or “in connection with via one or more intermediate elements or members.”

The foregoing description, for purposes of explanation, used specific nomenclature to provide a thorough understanding of the disclosure. However, it will be apparent to one skilled in the art that the specific details are not required in order to practice the systems and methods described herein. The foregoing descriptions of specific examples are presented for purposes of illustration and description. They are not intended to be exhaustive of or to limit this disclosure to the precise forms described. Many modifications and variations are possible in view of the above teachings. The examples are shown and described in order to best explain the principles of this disclosure and practical applications, to thereby enable others skilled in the art to best utilize this disclosure and various examples with various modifications as are suited to the particular use contemplated. It is intended that the scope of this disclosure be defined by the claims and their equivalents below.

1. A system for monitoring a patient, the system comprising:
a first drive electrode configured to be in contact with the patient, wherein the first drive electrode is configured to receive a first electrical current and inject it into the patient;
a first sense electrode configured to be in contact with the patient, wherein the first sense electrode is configured to sense a first bio-electric signal from the patient, wherein the first bio-electric signal is modified by the first electrical current;
an impedance circuit connected to the first drive electrode and the first sense electrode, wherein the impedance circuit is configured to measure a bio-impedance or bio-reactance waveform in response to the first bio-electric signal;
a physiological sensor connected to the first drive electrode and the first sense electrode, wherein the physiological sensor is configured to measure an electrocardiogram (ECG) waveform based upon the first bio-electric signal; and
a computing system configured to:
determine a respiration rate and/or a respiratory tidal volume of the patient based upon the bio-impedance or bio-reactance waveform; and
determine a heart rate and/or a heart rate variability of the patient based upon the ECG waveform.

2. The system of claim 1, further comprising:
a second drive electrode configured to be in contact with the patient, wherein the second drive electrode is configured to receive a second electrical current and inject it into the patient; and
a second sense electrode configured to be in contact with the patient, wherein the second sense electrode is configured to sense the first bio-electric signal and a second bio-electric signal from the patient, wherein the second bio-electric signal is modified by the second electrical current, and wherein the impedance circuit is also connected to the second drive electrode and the second sense electrode.

3. The system of claim 2, wherein the first and second electrical currents are received from the impedance circuit.

4. The system of claim 2, wherein the impedance circuit is configured to measure the bio-impedance or bio-reactance waveform in response to the first and second bio-electric signals.

5. The system of claim 2, wherein the physiological sensor is not configured to measure the ECG waveform based upon the second bio-electric signal.

6. The system of claim 1, further comprising a multiplex circuit connected to the impedance circuit and the physiological sensor, wherein the multiplex circuit is configured to switch between the impedance circuit and the physiological sensor.

7. The system of claim 1, wherein the computing system is further configured to analyze the bio-impedance or bio-reactance waveform to determine at least one of a stroke volume, a stroke volume variation, a cardiac output, a cardiac output variation, a cardiac index, a cardiac index variation, a fluid level, and a fluid responsivity corresponding to the patient.

8. The system of claim 1, wherein the computing system is further configured to analyze the bio-impedance or bio-reactance waveform to determine at least one of a respiration rate variability and a respiratory tidal volume variation corresponding to the patient.

9. The system of claim 1, further comprising an accelerometer configured to capture acceleration measurements corresponding to the patient, wherein the computing system is configured to determine a respiration rate and motion of the patient in response to the acceleration measurements.

10. The system of claim 1, wherein both the first drive electrode and the first sense electrode comprise an electrically conductive material, and wherein the electrically conductive material is one of a metal, a conductive fabric, a conductive foam, or a conductive elastomer.

11. A system for monitoring a patient wearing a diaper, comprising:
a flexible substrate configured to be worn between the diaper and the patient, the flexible substrate comprising:
a first drive electrode configured to be in contact with the patient, wherein the first drive electrode is configured to receive a first electrical current and inject it into the patient;
a second drive electrode configured to be in contact with the patient, wherein the second drive electrode is configured to receive a second electrical current and inject it into the patient;
a first sense electrode configured to be in contact with the patient, wherein the first sense electrode is configured to sense a first bio-electric signal from the patient, wherein the first bio-electric signal is modified by the first electrical current;
a second sense electrode configured to be in contact with the patient, wherein the second sense electrode is configured to sense the first bio-electric signal and a second bio-electric signal from the patient, wherein the second bio-electric signal is modified by the second electrical current;
an impedance circuit connected to the first and second drive electrodes and the first and second sense electrodes, wherein the impedance circuit is configured to measure a bio-impedance or bio-reactance waveform in response to the first and second bio-electric signals;
a physiological sensor connected to the first drive electrode and the first sense electrode, wherein the physiological sensor is configured to measure an electrocardiogram (ECG) waveform based upon the first bio-electric signal; and
a computing system configured to:
determine a respiration rate and/or a respiratory tidal volume of the patient based upon the bio-impedance or bio-reactance waveform; and
determine a heart rate and/or a heart rate variability of the patient based upon the ECG waveform.

12. The system of claim 11, wherein the flexible substrate is further configured so that the first and second drive electrodes and the first and second sense electrodes make physical contact with an abdomen, as opposed to a head, a chest, an arm, or a leg of the patient, such that no adhesive is needed to couple the first and second drive electrodes or the first and second sense electrodes to the patient.

13. The system of claim 11, wherein the flexible substrate further comprises:
a battery; and
an inductive coil configured to receive wirelessly transmitted frequencies and convert them into power used to recharge the battery.

14. The system of claim 13, wherein the flexible substrate comprises an elastomeric material, wherein the elastomeric material is configured to fully enclose the impedance circuit, the battery, and the inductive coil.

15. The system of claim 11, wherein the flexible substrate further comprises a securing mechanism configured to attach the flexible substrate to the diaper, and wherein the securing mechanism comprises one of an adhesive component and a magnet.

16. The system of claim 11, wherein the flexible substrate further comprises an optical sensor configured to capture an optical measurement, and wherein the computing system is configured to determine a heart rate of the patient in response to the optical measurement.

17. The system of claim 16, wherein the optical sensor comprises a photoplethysmogram (PPG) sensor including:
a red light source configured to emit red light;
an infrared light source configured to emit infrared light; and
a photodetector configured to measure an optical waveform in response to red light and the infrared light contacting the patient.

18. The system of claim 17, wherein the computing system is further configured to determine at least one of oxygen saturation and blood pressure based upon the optical waveform.

19. The system of claim 17, wherein the computing system is further configured to:
determine the heart rate of the patient based upon the optical waveform; and
compare the heart rate from the ECG waveform to the heart rate from the optical waveform to check for a false alarm.

20. The system of claim 11, wherein the flexible substrate further comprises an accelerometer that is configured to capture an acceleration measurement, and wherein the computing system is further configured to:
determine the respiration rate of the patient based upon the acceleration measurement; and
compare the respiration rate from the bio-impedance or bio-reactance waveform to the respiration rate from the acceleration measurement to check for a false alarm.
